(19) United States
(12) Patent Application Publication
(10) Pub. No.: US 2024/0404681 A1
(43) Pub. Date: Dec. 5, 2024

(54) SYSTEM, METHOD, AND COMPUTER PROGRAM PRODUCT FOR DISPLAYING AND IDENTIFYING ACTIONS FOR MEDICAL PROVIDERS

(71) Applicant: Lee Memorial Health System, Ft Myers, FL (US)

(72) Inventors: Chongsun Oh, Fort Myers, FL (US); Marcelo Zottolo, Cape Coral, FL (US)

(73) Assignee: Lee Memorial Health System, Ft. Myers, FL (US)

(21) Appl. No.: 18/326,317

(22) Filed: May 31, 2023

(51) Int. Cl. G16H 40/20; G16H 15/00

(52) U.S. Cl. CPC G16H 40/20; G16H 15/00

(57) ABSTRACT

A system for identifying actions for medical providers is disclosed. The system includes a computing device including memory storing program instructions for a data visualization tool and program instructions for a medical provider quality algorithm. The computing device includes at least one processor programmed or configured to execute the data visualization tool; receive data associated with medical providers; and execute the medical provider quality algorithm to generate a plurality of projected medical parameters and a medical provider quality rating. The system includes a database configured to store the data associated with medical providers, the plurality of projected medical parameters, and the medical provider quality rating. The data visualization tool is configured to generate a graphical display including a visual object corresponding to a parameter prioritization list. The visual object provides an indication of an identified action for a medical provider.

### FIELD

Embodiments relate to a system and method for generating projected medical parameters relating to performance, services, medical care, and/or systems of medical providers that may be ranked and measured such that the system and method may identify actions for medical providers in processes and/or systems of the medical providers such that values of the projected medical parameters may be influenced.

### BACKGROUND INFORMATION

In some instances, a medical provider (e.g., a hospital, a medical insurance provider, a clinic, etc.) may provide data relating to services of the medical provider to a data source based on one or more factors. For example, the medical provider may track a variety of metrics related to medical care of patients and the medical provider may provide the metrics to a government entity, a commercial entity, or another entity that may maintain the data in a data source controlled by the entity. The entity may collect data and/or metrics related to medical care from a plurality of different medical providers. The entity may collect the data on periodic basis and/or at the beginning of a selected period, such as annually, bi-annually, and/or the like.

The entity that collected the data related to medical care for the plurality of medical providers may later determine measures of performance of the plurality of medical providers using the data related to medical care. The entity may publish results of the measures of performance at a time after the data related to medical care is collected during the selected period (e.g., at the end of a year, and/or the like) such that the medical providers may view the results. However, the results are only published once per the selected period and are only available at a specific time when the results are published by the entity. Additionally, the results may not be useful to the plurality of medical providers if the results are published as raw measures of performance without specific guidance for improvements and/or without presenting the results in a graphical, image-based format.

Publishing and/or reporting results only once during the selected period causes inefficiencies in reporting measures of performance for medical providers. The published results may be further unhelpful not only by the temporal nature of results, but also by the lack of guidance and/or data analysis provided with raw results and/or raw measures of performance. The lack of guidance provided with the results as well as the temporal nature of the results creates inefficiencies because the results do not provide realtime insights or data analysis relating to a performance of a medical provider. Thus, any improvements by medical providers or in medical systems may lag and/or may not be implemented at all because the published results represent a snapshot rather than comprehensive, time-varying results. Additionally, medical providers must rely on receiving the published results at specific times, rather than receiving results of performance on-demand, when the medical provider requires analysis of its performance and/or systems. Additionally, the published results are not interactive such that the user may view and/or interact with the results in a useful format. The published results are simply raw data that may, to an untrained eye, be unhelpful and complicated, lacking any clarity or utility. A lack of an analysis of the results leaves medical providers without any direction, and a lack of realtime analysis leaves the published results practically useless. In some instances, medical providers may want to know exactly how they can change their care and/or their systems to improve their medical care and/or medical systems.

### SUMMARY

Embodiments may relate to a system for identifying actions for medical providers. The system may include a computing device including memory storing program instructions for a data visualization tool and program instructions for a medical provider quality algorithm. The computing device may include at least one processor programmed or configured to execute the data visualization tool. The processor may be further programmed or configured to receive data associated with medical providers. The processor may be further programmed or configured to execute the medical provider quality algorithm to generate a plurality of projected medical parameters and a medical provider quality rating. The system may also include a database configured to store the data associated with medical providers, the plurality of projected medical parameters, and the medical provider quality rating. The data visualization tool may be configured to generate a graphical display including a visual object corresponding to a parameter prioritization list based on a set of evaluation factors associated with a set of projected medical parameters of the plurality of projected medical parameters. The visual object providing an indication of an identified action for a medical provider.

Embodiments may relate to a computer-implemented method for identifying actions for medical providers. The method may include receiving data associated with medical providers. The method may further include generating a plurality of projected medical parameters including a parameter value based on the data associated with medical providers. A set of projected medical parameters of the plurality of projected medical parameters may be associated with a medical provider. The method may further include determining a medical provider quality rating associated with the medical provider based on the set of projected medical parameters and a plurality of action groups. Each projected medical parameter of the set of projected medical parameters may be assigned to an action group of the plurality of action groups. Each projected medical parameter may have a weight value based on a number of action groups that apply to the medical provider. The method may further include prioritizing each projected medical parameter based on an evaluation factor of a set of evaluation factors associated with the set of projected medical parameters and based on the parameter value to provide a parameter prioritization list. The parameter prioritization list may represent a generated recommendation of the set of projected medical parameters for a user to improve to increase the medical provider quality rating. The method may further include generating a graphical display associated with the medical provider for transmitting to a client display device. The graphical display may include a visual object corresponding to the parameter prioritization list. The visual object may provide an indication of an identified action for the medical provider.

### BRIEF DESCRIPTION OF THE DRAWINGS

Other objects and advantages of the present disclosure will become apparent to those skilled in the art upon reading the following detailed description of exemplary embodiments, in conjunction with the accompanying drawings, in which like reference numerals have been used to designate like elements, and in which:

FIG. 1 is a diagram of an exemplary system for identifying actions for medical providers as disclosed herein;

FIG. 2 is a flow diagram of an exemplary method for identifying actions for medical providers as disclosed herein;

FIGS. 3A-3E are diagrams of exemplary embodiments of an implementation of a process for identifying actions for medical providers as disclosed herein;

FIG. 4 is a diagram of an exemplary environment in which methods, systems, and/or computer program products, as disclosed herein, may be implemented according to some embodiments; and

FIG. 5 is a diagram of exemplary embodiments of components of one or more devices of FIG. 1 and/or FIG. 4 as disclosed herein.

### DETAILED DESCRIPTION

In accordance with exemplary embodiments of the present disclosure, a system and method for identifying actions for medical providers is provided. Embodiments may leverage publicly reported data associated with healthcare and/or medical providers and may provide for improved efficiency in reporting medical parameters (e.g., projected medical parameters) associated with healthcare and/or medical providers via a graphical display and/or dashboard application. The graphical display of medical parameters may be provided to users such that users (e.g., healthcare and/or medical provider personnel) may view medical parameters associated with their healthcare and/or medical provider in realtime. Embodiments may provide for improved graphical displays of reported medical parameters such that the user may view and/or interact with the medical parameters in a useful format (e.g., in a prioritized ranking). Some embodiments may allow users to view additional insights relating to the medical parameters such as insights related to medical parameters and data relationships that have been previously unexplored and have not been provided to healthcare and/or medical providers. Some embodiments may provide, for example via a graphical display, insights in realtime and/or may identify actions that medical providers may take to change a projected medical parameter such that a medical provider quality rating associated with the healthcare and/or medical provider may be improved. In this way, a healthcare and/or medical provider may take actions and or implement new systems and/or processes that may change a projected medical parameter. Taking action to change a projected medical parameter may help to improve the efficiency and quality of healthcare and/or medical provider systems and may reduce resources required to operate existing healthcare systems and processes.

Embodiments may provide graphical displays showing projected medical parameters for a healthcare and/or medical provider interested in projected medical parameters associated with the healthcare and/or medical provider. In some embodiments, graphical displays may be provided that show projected medical parameters for other healthcare and/or medical providers such that the healthcare and/or medical provider may view projected medical parameters for competitors and/or peer medical providers. Embodiments may provide roadmaps to improve quality of healthcare and/or medical provider systems based on the projected medical parameters and metadata of the projected medical parameters (e.g., a roadmap including a ranked prioritization of medical parameters).

Embodiments may provide for interactive graphical displays such that views of projected medical parameters can be customized based on the projected medical parameters and metadata of projected medical parameters. Embodiments of graphical displays may provide for analysis of trends of projected medical parameters, prioritized rankings of projected medical parameters, statistical distributions of projected medical parameters, comparisons of projected medical parameters across a plurality of medical providers associated with the project medical parameters, and geolocation maps displaying trends and associations of projected medical parameters. Along with the graphical displays, embodiments may provide for analysis and/or insights relating to the projected medical parameters such that the analysis and insights identify and/or cause actions that may improve healthcare and/or medical provider systems and/or processes such that a medical provider quality rating may be improved.

In this way, embodiments provide a novel approach to analyzing and displaying data associated with medical providers to allow medical providers to identify problem areas in realtime and to identify actions that may be taken immediately to remedy such problem areas. Embodiments may identify and/or cause specific actions to be taken to change (e.g., improve, increase, and/or the like) a projected medical parameter such that medical provider systems and/or processes may be improved in efficiency, use of resources, and/or effect. Such improvements in medical provider systems and/or processes may result in a change (e.g., improvement, increase) in a medical provider quality rating associated with the medical provider.

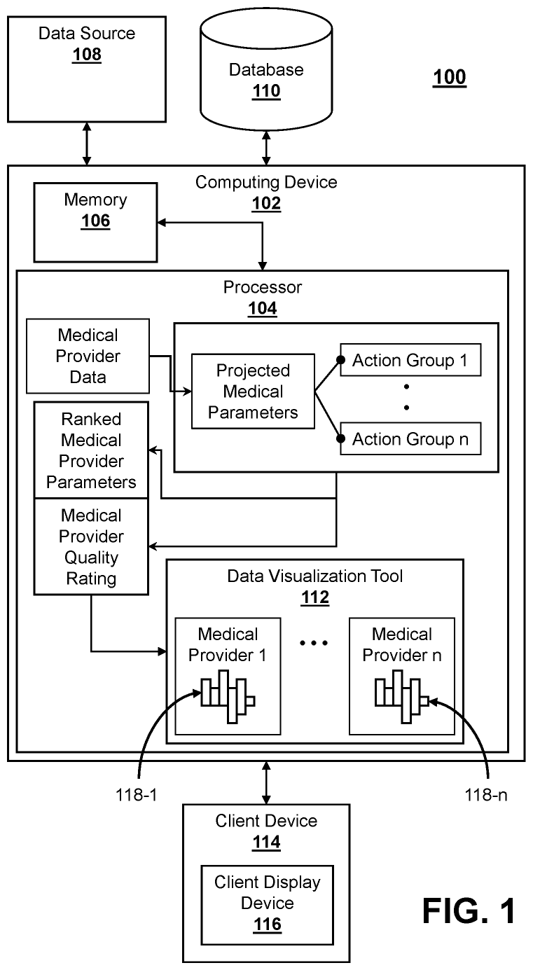

Referring to FIG. 1, shown is a diagram of an exemplary system 100 for identifying actions for medical providers. System 100 may include computing device 102, processor 104, memory 106, data source 108, database 110, and client device 114. Processor 104 may execute one or more software instructions such as data visualization tool 112 to generate visual object 118-1 to visual object 118-n (e.g., a plurality of visual objects, referred to individually as visual object 118 and collectively as visual objects 118 where appropriate) to be displayed on a display device.

Computing device 102 may include a computer, such as a server (e.g., a single server), a group of servers, and/or other like devices. Computing device 102 may include a processor (e.g., processor 104) and/or memory (e.g., memory 106) as described herein. Computing device 102 may include one or more client devices. Computing device 102 may include one or more servers and/or one or more client devices executing instructions (e.g., software instructions) that cause computing device 102 to perform one or more steps of methods as described herein. Computing device 102 may execute the one or more instructions via processor 104 and computing device 102 may store the one or more instructions in memory 106 for execution. In some embodiments, computing device 102 may include one or more computers, portable computers, laptop computers, tablet computers, mobile devices, cellular phones, wearable devices (e.g., watches, glasses, lenses, clothing, and/or the like), PDAs, and/or the like.

Processor 104 may include at least one processor (e.g., a multi-core processor), such as a central processing unit (CPU), an accelerated processing unit (APU), a graphics processing unit (GPU), a microprocessor, and/or the like. In some embodiments, processor 104 may include at least one processor having a single core (e.g., a single-core processor) or at least one processor having multiple cores (e.g., a multiprocessor, a multi-core processor, a processor including more than one core, and/or the like). Processor 104 may include at least one core that is a component of (e.g., part of) a single-core processor or a multiprocessor.

In some embodiments, processor 104 may be programmed to perform one or more steps of methods described herein. Processor 104 may execute one or more instructions (e.g., software instructions) that cause processor 104 to perform one or more steps of methods as described herein. Processor 104 may be in communication with memory 106 of computing device 102. Processor 104 may be capable of receiving information (e.g., data, data resources, and/or the like) from and/or communicating (e.g., transmitting) information to memory 106 of computing device 102. In some embodiments, processor 104 may be in communication with a separate memory 106 that is not associated with and/or is not included in computing device 102. Processor 104 may execute one or more instances of a data visualization tool (e.g., data visualization tool 112).

Memory 106 may include main memory internal to and/or associated with computing device 102. Memory 106 may include random access memory (RAM), read only memory (ROM), and/or another type of dynamic or static storage device (e.g., flash memory, magnetic memory, optical memory, etc.) that stores information and/or instructions for use by processor 104. In some non-limiting embodiments or aspects, memory 106 may include a storage component (e.g., a volatile storage component) that stores information and/or instructions for use by processor 104. In some non-limiting embodiments or aspects, memory 106 may include CPU memory of processor 104.

Memory 106 may store data and/or software instructions related to the operation and use of data visualization tool 112 and/or processor 104. For example, memory 106 may include a type of computer-readable medium. Memory 106 may transmit data to and/or receive data from processor 104. In some embodiments, memory 106 may be implemented by (e.g., part of) computing device 102 and/or processor 104. Alternatively, memory 106 may be a separate memory 106 not associated with and/or is not included in computing device 102.

Data source 108 may include a computing device, such as a server (e.g., a single server), a group of servers, and/or other like devices. In some embodiments, data source 108 may include one or more client devices. Data source 108 may include data which may be transmitted to computing device 102 and/or data that may be accessed and/or copied (e.g., downloaded) by computing device 102. In some embodiments, data source 108 may include one or more computers, portable computers, laptop computers, tablet computers, mobile devices, cellular phones, wearable devices (e.g., watches, glasses, lenses, clothing, and/or the like), PDAs, and/or the like. Data source 108 may be periodically updated with data (e.g., new data, newly collected data) that may be transmitted to computing device 102 and/or data that may be accessed by computing device 102.

Database 110 may include a computing device (e.g., a database device) configured to communicate with computing device 102 via a communication network. Database 110 may include a server, a group of servers, and/or other like devices. Database 110 may be associated with one or more computing devices providing interfaces such that a user (e.g., an administrative user) may interact with database 110 via the one or more computing devices. Database 110 may be in communication with computing device 102 such that database 110 is separate from computing device 102. In some embodiments, database 110 may be implemented by (e.g., may be part of) computing device 102. Database 110 may store one or more data records for use by computing device 102 and/or processor 104.

Data visualization tool 112 may include software instructions (e.g., a software application, a software program, and/or the like) configured to cause processor 104 to generate and/or provide graphical displays (e.g., visual objects 118) to a display device. Data visualization tool 112 may include one or more instances of data visualization tool 112 executing on processor 104 of computing device 102 and/or executing on other processors of other, separate computing devices, within a communication network. Data visualization tool 112 may include a plurality of visual objects 118 generated by processor 104 to display on a display device. Data visualization tool 112 may include software instructions configured to cause processor 104 to perform one or more steps of methods described herein. For example, data visualization tool 112 may include one or more instructions configured to cause processor 104 to perform one or more operations on data residing in memory 106 of computing device 102, such as transmitting data to a display device, formatting data for display on a display device, and/or the like. In some embodiments, data visualization tool 112 may be configured (e.g., via processor 104) to transmit the graphical display to at least one client device 114 including a client display device 116.

In some embodiments, data visualization tool 112 may execute on processor 104 (e.g., processor 104 may execute software instructions of data visualization tool 112) and data representing visual objects 118 and/or other graphical displays may be transmitted to client device 114 to be displayed by client display device 116. Alternatively, data visualization tool may execute on a processor of client device 114 and computing device 102 (e.g., processor 104 thereof) may transmit data to client device 114 for use with data visualization tool 112 executing on client device 114 such that client device 114 may generate graphical displays and/or visual objects 118 to display on client display device 116. In some embodiments, data visualization tool 112 may include a commercial-off-the shelf tool that may be installed on computing device 102 and executed by processor 104. In some embodiments, data visualization tool 112 may include a software application executing on a computing system and/or server separate from computing device 102. Data visualization tool 112 may include a cloud platform (e.g., a software service offered to client devices from a cloud and/or server environment) that may be accessed and/or used by computing system 102 and/or client device 114. In some embodiments, data visualization tool 112 may include a cloud platform such as those provided by Qlick® (e.g., Qlik Sense®, Qlik Cloud®, and/or the like).

Client device 114 may include one or more client-side devices and/or systems used to initiate or facilitate a network connection. As an example, a client device may include one or more computing devices used by a user, one or more personal computers used by a user, one or more mobile devices used by a user, and/or the like. In some embodiments, a client device may be an electronic device configured to communicate with one or more communication networks. For example, a client device may include one or more computers, portable computers, laptop computers, tablet computers, mobile devices, cellular phones, wearable devices (e.g., watches, glasses, lenses, clothing, and/or the like), PDAs, and/or the like. Moreover, a “client” may also refer to an entity (e.g., a user, a corporation, and/or the like) that owns, utilizes, and/or operates a client device.

Client device 114 may be configured to communicate with computing device 102 such that client device 114 may receive data (e.g., data representing visual objects 118) from computing device 102. Client device 114 may receive data to be transmitted to client display device 116 to generate graphical displays. In some embodiments, client device 114 may execute data visualization tool 112 to generate graphical displays and/or visual objects 118 based on data received from client device 102.

Client device 114 may include one or more client display devices 116. Client display device 116 may be integrated with (e.g., may be a part of) client device 114 or client display device 116 may be separate from client device 114. Client display device 116 may include a display device such as a screen, flat panel display, monitor, touchscreen, and/or other like devices. Client display device 116 may display and/or render data associated with data visualization tool 112 and or data associated with a plurality of visual objects 118. Client display device 116 may be used and/or viewed by a user such that the user may view visual objects 118 and other data displayed and/or rendered on client display device 116.

With continued reference to FIG. 1, embodiments may relate to a system 100 for identifying actions for medical providers. System 100 may include computing device 102. Computing device 102 may include memory 106 storing program instructions for data visualization tool 112 and storing program instructions for a medical provider quality algorithm. Computing device 102 may include at least one processor 104 programmed or configured to perform one or more operations and/or functions. For example, processor 104 may be programmed or configured to execute the data visualization tool 112. Processor 104 may be programmed or configured to receive data associated with medical providers from data source 108. For example, the data associated with medical providers may be received by computing device 102 (e.g., processor 104 thereof) from a medical provider data source, such as data source 108. Processor 104 may be programmed or configured to execute the medical provider quality algorithm to generate a plurality of projected medical parameters and a medical provider quality rating.

A projected medical parameter may include a type of projected medical parameter (e.g., a name of a projected medical parameter) and a parameter value. Thus, the plurality of projected medical parameters may include a plurality of types of projected medical parameters and a plurality of parameter values. An example of a projected medical parameter may include a type of projected medical parameter being “Surgical Site Infection from Colon Surgery” and may include a parameter value of 0.80. In some embodiments, a parameter value may include a decimal value, an integer value, a percentage value, a negative value, or any other suitable type of value.

Projected medical parameters generated by the system (e.g., system 100) may be generated based on the data associated with medical providers. In some instances, when a number of medical providers below a threshold of medical providers report data used to generate a particular projected medical parameter, then that particular projected medical parameter may not be generated by the system. For example, data associated with medical providers for a particular projected medical parameter may be reported and stored in data source 108 by less than one hundred (100) medical providers may be excluded and thus the particular projected medical parameters for the data that was reported by less than one hundred medical providers is not generated by the system. In this way, the system may only generate projected medical parameters for which the data associated with medical providers can be considered reliable such that reliable projected medical parameters may be generated. In some embodiments, other criteria for excluding (e.g., not generating) projected medical parameters may be used based on various factors and/or various properties of the data associated with medical providers that is collected.

System 100 may further include database 110 configured to store the data associated with medical providers, the plurality of projected medical parameters, and the medical provider quality rating generated by processor 104. Data visualization tool 112 (e.g., a computer-readable medium storing software instructions thereof) may be configured to cause processor 104 to generate a graphical display including visual objects 118. Data visualization tool 112 (e.g., via processor 104) may be configured to transmit the graphical display to at least one client device 114 including a client display device 116. Visual objects 118 may correspond to a list of prioritized projected medical parameters (e.g., a parameter prioritization list) displayed via the graphical display. Visual object 118 may provide (e.g., display) an indication of an identified action for the medical provider. For example, visual objects 118 may provide an indication that at least one projected medical parameter is associated with a greatest or least evaluation factor of the one or more evaluation factors.

The projected medical parameters of the parameter prioritization list may be prioritized and or displayed in a prioritized order based on one or more evaluation factors associated with each projected medical parameter in the parameter prioritization list. For example, each projected medical parameter may be associated with an evaluation factor of the one or more evaluation factors. In some embodiments, each of the one or more evaluation factors may include and/or may be associated with a value of standard deviation (e.g., a measure of standard deviation, a standardized score, etc.). An evaluation factor may include an absolute value or a signed value of a measure of standard deviation and/or a standardized score.

The evaluation factor may include a measure of standard deviation, a standardized score (e.g., a calculated z-score of each projected medical parameter), and the evaluation factor may include a measure of standard deviation or a standardized score having a signed value or an absolute value. In some embodiments, projected medical parameters associated with evaluation factors having a lower and/or least value may be assigned a higher priority (e.g., when the evaluation factor is a standardized score) and projected medical parameters associated with evaluation factors having a greater and/or greatest value may be assigned a lower priority. In some embodiments, projected medical parameters that are less than an average value of the projected medical parameter and are associated with an evaluation factor having a greater and/or greatest value, where the value of the evaluation factor is an absolute value (e.g., a positive value), may be assigned a higher priority (e.g., when the evaluation factor is an absolute value of a standardized score) and projected medical parameters that are greater than the average value of the projected medical parameter and are associated with an evaluation factor having a greater and/or greatest value, where the value of the evaluation factor is an absolute value, may be assigned a lower priority.

In some embodiments, projected medical parameters that are less than an average value of the projected medical parameter and are associated with an evaluation factor having a greater and/or greatest value may be assigned a higher priority (e.g., when the evaluation factor is a measure of standard deviation) and projected medical parameters that are greater than the average value of the projected medical parameter and are associated with an evaluation factor having a greater and/or greatest value, where the value of the evaluation factor is an absolute value, may be assigned a lower priority. In the case of a projected medical parameter where a lower parameter value is considered favorable (e.g., a value below the average value), then the projected medical parameter associated with an evaluation factor having a greater and/or greatest value may be assigned a lower priority (e.g., indicating that the parameter value is favorable when it is below the average value). This may apply similarly to a projected medical parameter that is greater than the average value and is associated with an evaluation factor having a value that is greater or greatest. The projected medical parameter would then be assigned a higher priority in that case when a lower parameter value is considered favorable.

In some embodiments, projected medical parameters may be prioritized based on one or more evaluation factors having a signed value. For example, a projected medical parameter may be associated with an evaluation factor having a signed value where a lower evaluation factor may be assigned a higher priority (e.g., readmissions, mortality). Alternatively, a projected medical parameter may be associated with an evaluation factor having a signed value where a greater evaluation factor may be assigned a higher priority (e.g., a percentage of healthcare workers vaccinated against influenza). To facilitate a combination of projected medical parameters associated with positively and/or negatively signed evaluation factors, standardization of evaluation factors may be used to ensure all projected medical parameters are on a common scale with a common direction. Therefore, evaluation factors may include a standardized score for each projected medical parameter by determining a standardized score (e.g., a z-score) for each projected medical parameter. In some embodiments, a signed value of an evaluation factor may be reversed (e.g., positive to negative or negative to positive) if necessary, such that the projected medical parameters may be prioritized based on greater evaluation factors being assigned a higher prioritization.

For example, the first, second, and third projected medical parameters may each be associated with evaluation factors equal to −1.2, 0.6, and 1.3, respectively. Processor 104 may rank and/or prioritize the first projected medical parameter as a first ranked parameter and/or may assign the first projected medical parameter a highest priority because the evaluation factor associated with the first projected medical parameter has a least value of the three evaluation factors (e.g., the evaluation factor associated with the first projected medical parameter indicates that the first projected medical parameter is furthest away from a mean and/or average value and a negative value of the evaluation factor indicates that the first projected medical parameter is below the mean and/or average value). In this way, the first projected medical parameter may be considered a top area of performance improvement and/or a highest priority.

Therefore, if the evaluation factor associated with a projected medical parameter is a signed value, not an absolute value, then the lower the value of the evaluation factor, the higher the projected medical parameter may be prioritized by processor 104, regardless of whether the projected medical parameter is below or above the mean and/or average value.

The at least one projected medical parameter being associated with the least or greatest evaluation factor of the one or more evaluation factors may represent that the at least one projected medical parameter is furthest from an average parameter value compared to the other projected medical parameters in a set of projected medical parameters. In this way, processor 104 (e.g., via data visualization tool 112) may display visual object 118 such that visual object 118 provides an indication of a projected medical parameter that may be improved. Thus, processor 104 may identify and/or generate one or more actions for the medical provider such that the medical provider may perform the one or more actions (e.g., by changing and/or updating medical processes and/or medical systems) may change the projected medical parameter so as to move (e.g., change) a parameter value associate with the projected medical parameter closer to the average parameter value. In this way, medical provider may improve the parameter values for projected medical parameters such that the parameter values reflect an average performance, or a performance that may be superior when compared to an average performance of peer medical providers.

Additionally, visual object 118 may provide an indication of an identified action for the medical provider by providing insights into the projected medical parameters based on the parameter values and by providing analysis of the parameter values of projected medical parameters. Processor 104 may generate new visual objects 118 based on comparisons of projected medical parameters between different medical providers, comparisons between projected medical parameters of one medical provider, or based on other suitable comparisons and/or analyses of the data associated with medical providers and the plurality of projected medical parameters.

As an example, a value of a first evaluation factor may determine a placement of a first projected medical parameter within the parameter prioritization list. As a further example, if a second evaluation factor has a greatest or least value of the one or more evaluation factors, then a second projected medical parameter associated with the second evaluation factor may be placed (e.g., displayed) in a first position in the parameter prioritization list. The first position in the parameter prioritization list may refer to a highest priority value in the parameter prioritization list, a top entry in the parameter prioritization list, an urgent entry in the parameter prioritization list, and/or the like.

In some embodiments, processor 104 may be programmed or configured to aggregate each projected medical parameter and each evaluation factor. For example, processor 104 may aggregate each projected medical provider and each evaluation factor associated with the projected medical parameters for a medical provider (e.g., a single medical provider of a plurality of medical providers). Each medical provider of a plurality of medical providers may be associated with a plurality of projected medical parameters and one or more evaluation factors specific to the medical provider.

In some embodiments, processor 104 may be programmed or configured to rank each projected medical parameter for the medical provider (e.g., the medical provider of a plurality of medical providers) based on the evaluation factor associated with each projected medical parameter and/or the parameter value associated with each projected medical parameter to provide ranked medical parameters. As an example, a medical provider may be associated with a plurality of projected medical parameters including a first projected medical parameter of heart failure 30-day mortality rate, a second medical parameter of excess days in acute care after hospitalization for heart failure, and a third projected medical parameter of responsiveness of hospital staff. The first projected medical parameter associated with the medical provider may have a first parameter value of 11.1 for the medical provider. The first projected medical parameter may be associated with a first average parameter value of 11.3 (the average parameter value being shared among the plurality of medical providers for the first projected medical parameter), and the first projected medical parameter may be associated with a first evaluation factor having a value of 0.12 for the medical provider. The second projected medical parameter associated with the medical provider may have a second parameter value of 5.8 for the medical provider. The second projected medical parameter may be associated with a second average parameter value of 4.7, and the second projected medical parameter may be associated with a second evaluation factor having a value of 0.42 for the medical provider. The third projected medical parameter associated with the medical provider may have a third parameter value of 4.0 for the medical provider. The third projected medical parameter may be associated with a third average parameter value of 3.28, and the third projected medical parameter may be associated with a third evaluation factor having a value of 0.69 for the medical provider.

With continued reference to the example described herein relating to the first, second, and third projected medical parameters, processor 104 may rank each projected medical parameter (e.g., the first, second, and third projected medical parameter) for the medical provider based on the evaluation factor and/or the parameter values associated with each projected medical parameter to provide ranked medical parameters. For example, processor 104 may rank the third projected medical parameter as a first ranked medical parameter because the third evaluation factor has a value that is greater than the first evaluation factor and the second evaluation factor. In some embodiments, processor 104 may rank each projected medical parameter based on evaluation factors being less than other evaluation factors for the medical provider to provide ranked medical parameters. In some embodiments, processor 104 may rank each projected medical parameter based on the parameter value and/or the average value of the projected medical parameter. For example, processor 104 may rank each projected medical parameter (e.g., the first, second, and third projected medical parameter) based on a magnitude of the parameter value and a difference between the parameter value and the average value. In some embodiments, ranking may be performed by processor 104 based on other factors, comparisons, and/or criteria related to the projected medical parameters, the parameter values associated with the projected medical parameters, and/or the evaluation factors associated with the projected medical parameters.

It should be understood that the plurality of projected medical parameters associated with the medical provider may include any number of different projected medical parameters and is not limited to the projected medical providers named herein. The aforementioned projected medical parameters (e.g., the first, second, and third projected medical parameters) are merely provided for the purposes of examples described herein.

Processor 104 may be further programmed or configured to prioritize the ranked medical parameters. For example, processor 104 may prioritize the ranked medical parameters based on the one or more evaluation factors. In an example where the one or more evaluation factors are absolute values, when prioritizing each ranked medical parameter, processor 104 may be programmed or configured to assign the ranked medical parameter a higher priority value when the evaluation factor associated with the ranked medical parameter is less than all remaining evaluation factors of the one or more evaluation factors and/or the parameter value of the ranked medical parameter is less than the average parameter value of the one or more projected medical parameters. In some embodiments, processor 104 may assign the ranked medical parameter a lower priority value when the evaluation factor associated with the ranked medical parameter is greater than all the remaining evaluation factors of the one or more evaluation factors and/or the parameter value of the ranked medical parameter is greater than the average parameter of the one or more projected medical parameters.

With continued reference to the example described herein relating to the first, second, and third projected medical parameters (and thus a first ranked medical parameter, a second ranked medical parameter, and a third ranked medical parameter), processor 104 may prioritize each of the ranked medical parameters based on the evaluation factor, the parameter values, and the average parameter value associated with each ranked medical parameter. For example, processor 104 may prioritize the ranked medical parameters (e.g., the first, second, and third projected medical parameters that were previously ranked). Processor 104 may compare the first evaluation factor having a value of 0.12 to each other evaluation factor (e.g., the second evaluation factor (0.42) and the third evaluation factor (0.69)) to determine which evaluation factor is the least evaluation factor (e.g., which evaluation factor has a least value of all evaluation factors of the set of evaluation factors). Processor 104 may determine that the first evaluation factor is the evaluation factor having the least value and/or is the least evaluation factor.

Additionally, processor 104 may prioritize each of the ranked medical parameters based on the parameter values and the average parameter value associated with each ranked medical parameter. For example, processor 104 may prioritize the ranked medical parameter (e.g., the first projected medical parameter that was previously ranked) based on a difference (e.g., an absolute difference or a signed difference) between the parameter value of the projected medical parameter and the average value of the projected medical parameter when the evaluation factor includes a measure of standard deviation.

Alternatively, processor 104 may prioritize each of the ranked medical parameters based on the evaluation factor associated with each ranked medical parameter. For example, processor 104 may prioritize the ranked medical parameter based on a least value of the evaluation factor for each projected medical parameters such that the projected medical parameters are prioritized from least evaluation factor (having a highest priority) to greatest evaluation factor (having a lowest priority) when the evaluation factor includes a standardized score.

Processor 104 may prioritize the ranked medical parameters by comparing the differences between the parameter value of the projected medical parameter and the average value of the projected medical parameter (e.g., parameter value-average parameter value) for each ranked medical parameter and prioritizing the ranked medical parameter based on a magnitude of the difference (e.g., an absolute difference) and/or a direction of the parameter value from the average parameter value (e.g., a signed difference). In some embodiments, processor 104 may prioritize the ranked medical parameters based on a highest ranking in the ranked medical parameters (e.g., highest ranking may equate to the projected medical parameter having the greatest evaluation factor) when compared to other ranked medical parameters that have not yet been prioritized (e.g., remaining ranked medical parameters and/or remaining evaluation factors not yet compared).

Thus, the process of prioritization carried out by processor 104 may proceed as follows where the evaluation factor is a measure of standard deviation. Processor 104 may determine a first difference between the first parameter value of the first projected medical parameter having a value of 11.1 and the first average parameter value of 11.3 (e.g., a first absolute difference of 0.2 and a first signed difference of −0.2). Processor 104 may determine a second difference between the second parameter value of the second projected medical parameter having a value of 5.8 and the second average parameter value of 4.7 (e.g., a second absolute difference of 1.1 and a second signed difference of 1.1). Processor 104 may determine a third difference between and the third parameter value of 4.0 of the third projected medical parameter and the third average parameter value of 3.28 (e.g., a third absolute difference of 0.72 and a third signed difference of 0.72).

Similarly, where the evaluation factor is a standardized score, the projected medical parameters may be prioritized based on the value of the evaluation factor (e.g., standardized score), starting with the least evaluation factor as the highest priority.

As shown in the examples described herein, the current ranking of the ranked medical parameters may be based on a value of the evaluation factors where the evaluation is a standardized score, thus resulting in the ranked medical parameters representing the following:
[Rank 1. First projected medical parameter (evaluation factor=0.12),
Rank 2. Second projected medical parameter (evaluation factor=0.42),
Rank 3. Third projected medical parameter (evaluation factor=0.69)].

Although ranking and prioritizing by processor 104 may result in a projected medical parameter being ranked and prioritized in the same position (e.g., first, second, third, etc.), this is not always the case, and this disclosure should not be limited to such ranking and/or prioritizing. Ranking and/or prioritizing a projected medical parameter in a same position may be due to the fact that ranking may be performed based on any number of factors, including based on a magnitude of a parameter value associated with the projected medical parameters, a magnitude of a percentile value associated with the projected medical parameters, a magnitude of a normalized value associated with the projected medical parameters, a magnitude of an evaluation factor associated with the projected medical parameters, and/or any combination thereof.

In some embodiments, processor 104 may be further programmed or configured to determine an average parameter value. For example, processor 104 may determine the average parameter value based on averaging each parameter value of each projected medical parameter across a plurality of medical providers. That is, processor 104 may collect (e.g., identify, select, and/or the like) a first set of parameter values, where the first set of parameter values is associated with a first medical provider. Processor 104 may collect a second set of parameter values, where the second set of parameter values is associated with a second medical provider. Processor 104 may collect n sets of parameter values, each set of parameter values of the n sets of parameter values being associated with a medical provider of n medical providers. Processor 104 may determine the average parameter value by summing a projected medical parameter from each set of projected medical parameters associated with each of the n medical providers to provide a summed parameter value, where the projected medical parameter from each set of projected medical parameters corresponds across the medical providers. Processor 104 may then divide the summed parameter value by n, or a total number of medical providers included in a determination of the average parameter value. Determination of an average parameter value may apply to one type of projected medical parameter across the plurality of medical providers.

As a further example, a type of projected medical parameter may include a parameter associated with catheter-associated urinary tract infections. Each medical provider of the plurality of medical providers may include a parameter value associated with the parameter associated with catheter-associated urinary tract infections. There may exist a first medical provider having a first parameter value of 0.45 associated with catheter-associated urinary tract infections. A second medical provider may have a second parameter value of 0.24 associated with catheter-associated urinary tract infections and a third medical provider may have a third parameter value of 0.6 associated with catheter-associated urinary tract infections. In order to determine the average parameter value associated with catheter-associated urinary tract infections across medical providers, processor 104 may sum the first parameter value, the second parameter value, and the third parameter value to provide a summed parameter value of 1.29. Processor 104 may then divide the summed parameter value by a number of medical providers (in this example, three (3) medical providers) included in the determination of the average value to determine the average parameter value to be 0.43.

It should be understood that each medical provider of the plurality of medical providers associated with the plurality of projected medical parameters will each have a separate set of parameter values and a separate set of evaluation factors associated with the plurality of projected medical parameters, but each medical provider may share each average parameter value associated with the plurality of projected medical parameters. For example, a first medical provider may be associated with a first, second, and third projected medical parameter having parameter values of 0.2, 0.5, and 0.7 respectively and evaluation factors 0.02, 0.13, and 0.22 respectively. A second medical provider may be associated with the same first, second, and third projected medical parameters having parameter values of 0.1, 0.9, and 0.6 respectively and evaluation factors 0.011, 0.06, and 0.15 respectively. Thus, parameter values and evaluation factors are specific to medical providers. Each medical provider will have its own parameter values and evaluation factors for each projected medical parameter that the medical provider is associated with, while each medical provider may be associated with the same projected medical parameters. Further, both the first and second medical provider associated with the first, second, and third projected medical parameter may share an association with each average parameter value for each projected medical parameter. For example, the first, second, and third projected medical parameter may have average parameter values of 0.75, 0.42, and 0.53 respectively. Each medical provider associated with the first, second, and third projected medical parameter will share the average parameter values of 0.75, 0.42, and 0.53 because the average parameter values are based on the parameter values across all medical providers.

In some embodiments, processor 104 may be further programmed or configured to identify a contact point associated with the at least one projected medical parameter. A contact point may include one or more clinical personnel and/or one or more business personnel responsible for the at least one projected medical parameter. In some embodiments, a contact point may include a system (e.g., a computing system) associated with the projected medical parameter that is part of a medical provider. For example, a medical provider may own, operate, and/or control a computing system (e.g., a contact computing system) associated with a projected medical parameter. The contact computing system may collect and/or store data associated with the projected medical parameter for the medical provider. The contact computing system may provide a contact point associated with the projected medical parameter for the medical provider in that messages, requests, and/or the like may be transmitted to the contact computing system to request data associated with the projected medical parameter. Additionally, messages, signals, and/or other data may be transmitted to the contact computing system for generating an alert, requesting a response, and/or requesting an action from the contact computing system and/or a user of the contact computing system.

The number and arrangement of systems and devices shown in FIG. 1 are provided as an example. There may be additional systems and/or devices, fewer systems and/or devices, different systems and/or devices, and/or differently arranged systems and/or devices than those shown in FIG. 1. Furthermore, two or more systems or devices shown in FIG. 1 may be implemented within a single system or device, or a single system or device shown in FIG. 1 may be implemented as multiple, distributed systems or devices. Additionally or alternatively, a set of systems (e.g., one or more systems) or a set of devices (e.g., one or more devices) of system 100 may perform one or more functions described as being performed by another set of systems or another set of devices of system 100.

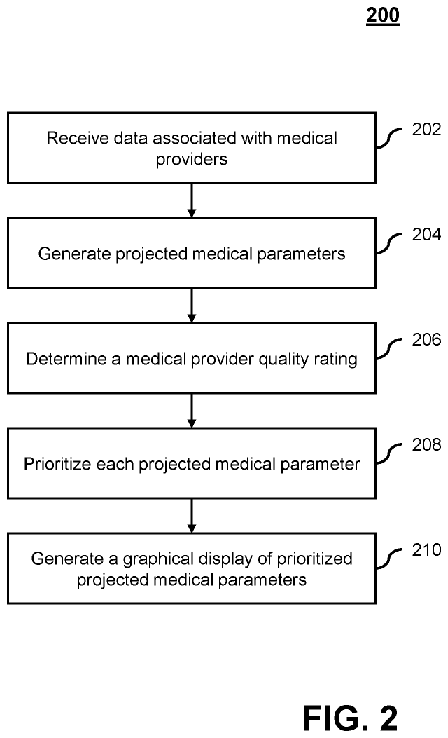

Referring to FIG. 2, shown is a flow diagram of an exemplary method 200 for identifying actions for medical providers according to embodiments as disclosed herein. The steps shown in FIG. 2 are for example purposes only. It will be appreciated that additional, fewer, different, and/or a different order of steps may be used in some embodiments.

As shown in FIG. 2, at step 202, method 200 may include receiving data associated with medical parameters. For example, processor 104 may receive data associated with medical providers from memory 106, data source 108, and/or database 110. In some embodiments, processor 102 may receive the data associated with medical providers from a medical provider data source. The medical provider data source may collect the data associated with medical providers from a plurality of medical providers that report the data associated with medical providers (e.g., data related to medical services, medical statistics, data used to generate projected medical parameters, and/or the like). In some embodiments, processor 102 may receive the data associated with medical providers automatically (e.g., via a push, an automatic download, and/or the like) from a medical provider data source (e.g., data source 108).

The plurality of medical providers may include at least one medical provider associated with a set of data in the data associated with medical providers. Processor 104 may use the set of data to generate a set of projected medical parameters associated with the medical provider. Each other medical provider of the plurality of medical providers may be associated with a different set of data in the data associated with medical providers (e.g., and a different set of projected medical parameters). For example, a medical provider of the plurality of medical providers may be associated with a set of data in the data associated with medical providers where the set of data was provided by the medical provider to the medical provider data source (e.g., data source 108).

In some embodiments, processor 104 may generate a data profile for the data associated with medical providers. For example, processor 104 may create a plurality of data profiles for a plurality of data files of the data associated with medical parameters. A relationship between a data profile and a data file may be a one-to-one relationship, for example, one data profile may be generated for one data file of the plurality of data files of the data associated with medical providers. In some embodiments, a data profile may include data types of the data associated with medical providers. A data profile may allow for automatic preparation of the data associated with medical providers. For example, a data profile may facilitate automatic conversion of data types, automatic preparation of data columns such that the data associated with medical providers may be cleaned and prepared to be input into a machine learning model, an algorithm, or other suitable system. In this way, computing device 102 (e.g., processor 104 thereof) may format the data associated with medical providers automatically and may reduce time and resources required to format the data associated with medical providers when the structure of the data associated with medical providers does not change from one dataset to another.

In some embodiments, processor 104 may clean, filter, and/or adjust each data profile. For example, processor 104 may use one or more data frames (e.g., R data frame, pandas DataFrame, and/or the like) and/or one or more data frame functions to perform cleansing, row filtering, and/or any other suitable adjusts to a data profile and/or the data associated with medical providers. In this way, processor 104 may prepare the plurality of data profiles for inputting into a machine learning model, algorithm, and/or other suitable system for processing. As further examples, processor 104 may use one or more data frames to change column names, add columns, or perform conversions of data types. In some embodiments, each data frame may be saved and/or stored in database 110.

In some embodiments, processor 104 may import each data file into a database. For example, processor 104 may import each data file into database 110 using a specified data frame of the one or more data frames and/or a specified data frame function of the one or more data frame functions. Processor 104 may iterate through the plurality of data files to import each data files into database 110. Processor 104 may check whether a data file has previously been imported into database 110, and If the data file has previously been imported into database 110 (e.g., the data file exists in database 110), processor 104 may ignore and/or skip the data file while iterating through the plurality of data files. In some embodiments, processor 104 may insert data of each data file of the plurality of data files into a single table such that the data may be reported by processor 104.

In some embodiments, processor 104 may generate a view (e.g., a database view, a subset of a database, and/or the like) of the data associated with medical providers in database 110. For example, processor 104 may create a view of database 110 that includes previously reported data associated with medical providers, previously generated projected medical parameters, and/or previously generated medical provider quality ratings for each medical provider of the plurality of medical providers.

As shown in FIG. 2, at step 204, method 200 may include generating projected medical parameters. For example, processor 104 may generate a plurality of projected medical parameters associated with at least one medical provider based on the data associated with medical providers. Processor 104 may generate a plurality of projected medical parameters, where the plurality of projected medical parameters includes one or more sets of projected medical parameters (e.g., n sets of projected medical parameters, where n represents an integer). Each of the one or more sets of projected medical parameters may be associated with a medical provider of a plurality of medical providers. For example, if there are n medical providers in the plurality of medical providers, then the plurality of projected medical parameters may include n sets of projected medical parameters, where each set of projected medical parameters is associated with a medical provider. In this way, a projected medical parameter may be represented across each medical provider of the plurality of medical providers.

As an example, the plurality of medical providers may include n=4 medical providers. The plurality of projected medical parameters may include n=4 sets of projected medical parameters, where each set of projected medical parameters may include 8 projected medical parameters. In this way, each projected medical parameter in each set of projected medical parameters may represent a type of projected medical parameter and a parameter value for each medical provider. For example, Table 1 below represents a plurality of projected medical parameters including n=4 sets of 8 projected medical parameters associated with n=4 medical providers. As shown in Table 1, each projected medical parameter has a parameter value that is associated with a medical provider across all medical providers. It should be understood that the number of projected medical providers in each set of projected medical parameters is not limited to 8 and the number of medical providers is not limited to 4. These numbers are used purely for example.

TABLE 1
Medical Providers Associated with Projected Medical Parameters

| Projected Medical | | | | |
|---|---|---|---|---|
| Parameters/Medical | Medical | Medical | Medical | Medical |
| Provider | Provider 1 | Provider 2 | Provider 3 | Provider 4 |
| Projected Medical | Parameter | Parameter | Parameter | Parameter |
| Parameter 1 (e.g., Acute | value = 11.6% | value = 5.5% | value = 10.1% | value = 9.2% |
| Myocardial Infarction 30- | | | | |
| Day Mortality Rate) | | | | |
| Projected Medical | Parameter | Parameter | Parameter | Parameter |
| Parameter 2 (e.g., Heart | value = 13.4% | value = 11.0% | value = 9.9% | value = 10.8% |
| Failure 30-Day Mortality | | | | |
| Rate) | | | | |
| Projected Medical | Parameter | Parameter | Parameter | Parameter |
| Parameter 3 (e.g., | value = 14.7% | value = 13.1% | value = 12.2% | value = 12.8% |
| Pneumonia 30-Day | | | | |
| Mortality Rate) | | | | |
| Projected Medical | Parameter | Parameter | Parameter | Parameter |
| Parameter 4 (e.g., Excess | value = −5.8 | value = 7.1 | value = −4.6 | value = 3.2 |
| Days in Acute Care after | | | | |
| Hospitalization for Heart | | | | |
| Failure) | | | | |
| Projected Medical | Parameter | Parameter | Parameter | Parameter |
| Parameter 5 (e.g., Hospital | value = 1.0 | value = 2.1 | value = 0.8 | value = 1.3 |
| Visits after Hospital | | | | |
| Outpatient Surgery) | | | | |
| Projected Medical | Parameter | Parameter | Parameter | Parameter |
| Parameter 6 (e.g., | value = 0.92 | value = 0.81 | value = 0.90 | value = 0.94 |
| Catheter-Associated | | | | |
| Urinary Tract Infection) | | | | |
| Projected Medical | Parameter | Parameter | Parameter | Parameter |
| Parameter 7 (e.g., | value = 2.00 | value = 3.00 | value = 1.50 | value = 2.00 |
| Communication with | | | | |
| Doctors) | | | | |
| Projected Medical | Parameter | Parameter | Parameter | Parameter |
| Parameter 8 (e.g., | value = 86% | value = 91% | value = 89% | value = 82% |
| Healthcare Personnel | | | | |
| Influenza Vaccination) | | | | |

As shown in Table 1, a parameter value of a projected medical parameter may vary in format and/or magnitude based on a type of projected medical parameter. For example, some projected medical parameters may have parameter values that include positive values, negative values, a percentage, a decimal value, an integer, and/or the like.

As shown in FIG. 2, at step 206, method 200 may include determining a medical provider quality rating. For example, processor 104 may determine a medical provider quality rating based on the projected medical parameters and a plurality of action groups. The medical provider quality rating may be associated with a medical provider. For example, a medical provider may be associated with (e.g., assigned) a medical provider quality rating that was determined based on the projected medical parameters (e.g., the set of projected medical parameters of the plurality of projected medical parameters) that are associated with the medical provider. As a further example, referring to Table 1, medical provider 1 may be associated with a medical provider quality rating where the medical provider quality rating was determined based on the eight (8) projected medical parameters (e.g., parameter values) in the column for medical provider 1. Thus, processor 104 may determine a medical provider quality rating based on a set of projected medical parameters that are associated with the medical provider.

In some embodiments, each projected medical parameter (e.g., each projected medical parameter in a set of projected medical parameters and/or in the plurality of projected medical parameters) may be associated with (e.g., assigned to) an action group of a plurality of action groups. For example, a first medical provider may have three (3) action groups associated with the first medical provider, such as a mortality action group, a safety of care action group, and a patient experience action group. Projected medical parameters may be assigned to each action group based on a type of the projected medical parameter corresponding to the action group. A projected medical parameter of communication with doctors, as shown in Table 1, may be assigned to the patient experience action group.

In some embodiments, each action group (e.g., each projected medical parameter assigned to an action group) may be associated with a weight value based on a number of action groups that apply to the medical provider. In some embodiments, the number of action groups that apply to the medical provider may be predetermined. In some embodiments, the number of action groups (and the name and/or type of action group) that may apply to the medical provider may be determined based on services provided by the medical provider and/or actions performed by the medical provider. In some embodiments, processor 104 may assign an action group of a medical provider a weight value of 0% if the medical provider does not report any data (e.g., data associated with medical providers) that may allow processor 104 to generate any projected medical parameters for that action group. For example, if a medical provider does not report any data that may generate projected medical parameters that would be assigned to a mortality action group, then the medical provider may have a weight value of 0% assigned to the mortality action group (e.g., a weight value of 0% may be assigned to each projected medical parameter in the mortality action group).

As shown in FIG. 2, at step 208, method 200 may include prioritizing each projected medical parameter. For example, processor 104 may prioritize each projected medical parameter to provide a parameter prioritization list. In some embodiments, the parameter prioritization list may represent a generated recommendation of one or more projected medical parameters associated with the medical provider for a user to improve in order to increase the medical provider quality rating. For example, the parameter prioritization list may include a list of a set of projected medical parameters and associated parameter values for a medical provider where the parameters are prioritized in order of medical parameters that may need to be improved by the medical provider.

In some embodiments, processor 104 may prioritize the projected medical parameters based on an evaluation factor. An evaluation factor may be associated with a (e.g., one) projected medical parameter of the plurality of projected medical parameters such that there exists a plurality of evaluation factors including sets of evaluation factors, where each set of evaluation factors is associated with a medical provider. In some embodiments, processor 104 may generate an evaluation factor for each projected medical parameter of the plurality of projected medical parameters.

In some embodiments, processor 104 may prioritize each projected medical parameter based on a parameter value of each projected medical parameter. Each projected medical parameter of the plurality of projected medical parameters may be associated with a different parameter value for each medical provider (e.g., see Table 1).

In some embodiments, processor 104 may prioritize each projected medical parameter by aggregating each projected medical parameter and each evaluation factor associated with the projected medical parameter. For example, processor 104 may aggregate each projected medical parameter of a set of projected medical parameters associated with the medical provider and each evaluation of a set of evaluation factors associated with the medical providers such that the set of projected medical parameters associated with the medical provider and the set of evaluation factors associated with the medical provider are separated from the data associated with medical providers (e.g., the remaining data associated with medical providers) so that the set of projected medical parameters and the set of evaluation factors can be processed (e.g., operated on separately, stored in separate memory locations, and/or the like). In some embodiments, processor 104 may aggregate each projected medical parameter of a set of projected medical parameters associated with the medical provider and each evaluation of a set of evaluation factors associated with the medical providers such that the set of projected medical parameters associated with the medical provider and the set of evaluation factors associated with the medical provider are displayed on a graphical display by data visualization tool 112 as separate from other data that is displayed (e.g., in a separate graph, separate presentation, and/or the like).

Processor 104 may further prioritize each projected medical parameter by ranking each projected medical parameter for the medical provider based on the evaluation factor to provide ranked medical parameters. Processor 104 may rank each projected medical parameter of the set of projected medical parameters associated with the medical provider based on any suitable factor, such as the evaluation factor, the parameter value, the type of projected medical parameter, an amount of data associated with medical providers that is associated with the set of projected medical parameters that was previously received by computing device 102 from data source 108, and/or any other suitable factor that may provide a mechanism for processor 104 to rank the projected medical parameters of a set of projected medical parameters.

In some embodiments, processor 104 may prioritize each ranked medical parameter of the ranked medical parameters by assigning the ranked medical parameter a higher priority value (e.g., a higher priority integer, assigning the ranked medical provider a lower integer such that the ranked medical parameter is placed at the beginning of a prioritized list of ranked medical parameters, and/or the like). For example, processor 104 may assign a ranked medical parameter a higher priority value where the parameter value of the ranked medical parameter is greater than or less than the average parameter value (or greater than the average parameter value when a negative parameter value is favored) of the one or more projected medical parameters (e.g., the parameter values of the projected medical parameters considered by processor 104 when determining the average parameter value) and/or where the evaluation factor associated with the ranked medical parameter is less than all remaining evaluation factors of the one or more evaluation factors associated with all remaining ranked medical parameters (e.g., ranked medical parameters that have not yet been prioritized by processor 104).

Processor 104 may assign the ranked medical parameter a lower priority value where the parameter value of the ranked medical parameter is greater than or less than the average parameter value of the one or more projected medical parameters and/or where the evaluation factor associated with the ranked medical parameter is greater than all the remaining evaluation factors of the one or more evaluation factors associated with all the remaining ranked medical parameters (e.g., ranked medical parameters that have not yet been prioritized by processor 104).

In some embodiments, processor 104 may prioritize each ranked medical parameter of the ranked medical parameters by assigning the ranked medical parameter a higher priority value (e.g., a higher priority integer, assigning the ranked medical provider a lower integer such that the ranked medical parameter is placed at the beginning of a prioritized list of ranked medical parameters, and/or the like) where the parameter value of the ranked medical parameter is greater than the average parameter value (e.g., when a lower parameter value indicates a better performance) of the one or more projected medical parameters (e.g., the parameter values of the projected medical parameters considered by processor 104 when determining the average parameter value) and/or where the evaluation factor associated with the ranked medical parameter is greater than all remaining evaluation factors of the one or more evaluation factors associated with all remaining ranked medical parameters (e.g., ranked medical parameters that have not yet been prioritized by processor 104).

Processor 104 may assign the ranked medical parameter a lower priority value where the parameter value of the ranked medical parameter is less than the average parameter value of the one or more projected medical parameters (e.g., when a lower parameter value indicates a better performance) and/or where the evaluation factor associated with the ranked medical parameter is greater than all the remaining evaluation factors of the one or more evaluation factors associated with all the remaining ranked medical parameters (e.g., ranked medical parameters that have not yet been prioritized by processor 104). In some embodiments, a signed parameter value of the ranked medical parameter may be reversed (e.g., positive to negative or negative to positive) before an evaluation score (e.g., standardized score) is determined such that when ranking and/or prioritizing parameters, greater parameter values can be provided a lower rank and/or prioritization regardless of the context of the projected medical parameter (e.g., where a negative value of a projected medical parameter may indicate poor performance). In this way, the system may properly rank and/or prioritize projected medical parameters even where lower and/or higher parameter values indicate poor performance.

Considering Table 1 and taking medical provider 1 as an example, with an example set of eight (8) projected medical parameters (e.g., the same projected medical parameters shown in Table 1), processor 104 may determine an average parameter value for each projected medical parameter according to Table 2 (based on the four (4) medical providers shown in Table 1 and medical provider 1 shown in Table 2).

TABLE 2
Projected Medical Parameters for Medical Provider 1, Average
Parameter Value, and Evaluation Factor for Medical Provider 1

| | | | Evaluation |
|---|---|---|---|
| | | Average | Factors for |
| Projected Medical Parameters/ | Medical | Parameter | Medical Provider |
| Medical Provider | Provider 1 | Value | 1 |
| Projected Medical Parameter 1 | Parameter | 9.48% | 0.01 |
| (e.g., Acute Myocardial Infarction | value = 11.6% | | |
| 30-Day Mortality Rate) | | | |
| Projected Medical Parameter 2 | Parameter | 11.275% | 0.02 |
| (e.g., Heart Failure 30-Day | value = 13.4% | | |
| Mortality Rate) | | | |
| Projected Medical Parameter 3 | Parameter | 13.2% | 0.02 |
| (e.g., Pneumonia 30-Day Mortality | value = 14.7% | | |
| Rate) | | | |
| Projected Medical Parameter 4 | Parameter | 1.18 | 0.71 |
| (e.g., Excess Days in Acute Care | value = 6.0 | | |
| after Hospitalization for Heart | | | |
| Failure) | | | |
| Projected Medical Parameter 5 | Parameter | 1.12 | 0.18 |
| (e.g., Hospital Visits after Hospital | value = 0.4 | | |
| Outpatient Surgery) | | | |
| Projected Medical Parameter 6 | Parameter | 0.888 | 0.59 |
| (e.g., Catheter-Associated Urinary | value = 0.87 | | |
| Tract Infection) | | | |
| Projected Medical Parameter 7 | Parameter | 1.85 | 1.04 |
| (e.g., Communication with | value = 0.75 | | |
| Doctors) | | | |
| Projected Medical Parameter 8 | Parameter | 87% | 1.2 |
| (e.g., Healthcare Personnel | value = 86% | | |
| Influenza Vaccination) | | | |

Processor 104 may also determine a set of evaluation factors for medical provider 1 as shown in Table 2. Assuming the projected medical parameters for medical provider 1 are ranked as shown in Table 2 (e.g., as ranked medical parameters), processor 104 may prioritize the ranked medical parameters by assigning a priority of one (1) to projected medical parameter 4 (e.g., Excess Days in Acute Care after Hospitalization for Heart Failure). For projected medical parameter 4, the parameter value for medical provider 1 is greater than the average parameter value, however this projected medical parameter indicates a better performance as values are lower (e.g., because a number of excess days in acute care is considered unfavorable performance as the number of days increases, thus a lower number is considered as better performance). Thus, the parameter value for projected medical parameter 4 is higher than the average and is considered as a low performing projected medical parameter for medical provider 1. Additionally, projected medical provider 1 is assigned a priority of one (1) because, while the parameter value indicates lower performance, projected medical parameter 8 also has a least evaluation factor of 0.01.

In some embodiments, the evaluation factor may indicate a standard deviation of the projected medical parameter across medical providers. Alternatively, the evaluation factor may indicate a distance (e.g., a numerical distance) between the parameter value and the average parameter value. For example, the evaluation factor for projected medical parameter 4 of medical provider 1 may indicate a standard deviation value of 24.85. Alternatively, the evaluation factor for projected medical parameter 4 (e.g., specifically for medical provider 1) may include a difference between the parameter value (e.g., 6.0) and the average parameter value (e.g., 1.18) therefore equaling a value of 4.82. Alternatively, the evaluation factor for projected medical parameter 4 may include a standardized score (e.g., a number of standard deviations from a mean, a z-score, and/or the like) such as shown in Table 2 (e.g., 0.71). In this way, processor 104 may use the evaluation factor (e.g., standardized score) of a projected medical parameter to rank and/or prioritize the projected medical parameter.

In this example, projected medical parameter 8 has an associated evaluation factor having a greatest value, where the evaluation factor is a standardized score (e.g., a greater value indicates favorable performance), and thus is assigned a lower (e.g., a lowest) priority value of one (8).

Continuing with the example considering Table 2 where each evaluation factor is a standardized score, projected medical parameter 1 may be assigned a priority of one (1) because the parameter value for projected medical parameter 1 is greater than the average parameter value, where a higher parameter value indicates unfavorable performance. Projected medical parameter 1 has a least evaluation factor of all the remaining evaluation factors (e.g., all evaluation factors except for the evaluation factor for projected medical parameter 8, because projected medical parameter 8 has already been prioritized). Projected medical provider 2 or 3 could be assigned a priority of two (2) because projected medical provider 2 has a next lowest evaluation factor. Since the parameter value of projected medical parameter 3 is further from the average value, projected medical parameter 3 may be assigned a priority of three (3).

In the following example, a parameter prioritization list may be provided as (1) projected medical parameter 1 (e.g., because projected medical parameter 1 is associated with an evaluation factor having a least value), (2) projected medical parameter 3, (3) projected medical parameter 2, (4) projected medical parameter 5, (5) projected medical parameter 6, (6) projected medical parameter 4, (7) projected medical parameter 7, and (8) projected medical parameter 8 (e.g., because projected medical parameter is associated with an evaluation factor having a greatest value). Following the example, projected medical parameter 8 is assigned the lowest priority value of eight (8) because projected medical parameter 8 is associated with an evaluation factor (e.g., a standardized score) having a greatest value. In this way, a parameter prioritization list may be generated for each medical provider. The aforementioned example uses an evaluation factor including a standardized score. However, projected medical parameters may be ranked and/or prioritized similarly or differently when the evaluation factor used is a measure of standard deviation, a difference value, or another value.

Although the aforementioned example is provided for generating a parameter prioritization list, other parameter prioritization lists may be comprehended based on different evaluation factors determined for each projected medical parameter (e.g., a difference value, a normalized value, a standardized value, and/or the like).

As shown in FIG. 2, at step 210, method 200 may include generating a graphical display of prioritized projected medical parameters. For example, processor 104 may generate (e.g., via data visualization tool 112) a graphical display associated with the medical provider that may be transmitting to a client display device. Processor 104 may transmit the graphical display to client device 114. Alternatively, processor 104 may transmit data associated with the graphical display to client device 114 such that client device 114 may generate the graphical display and display the graphical display via client display device 116. The graphical display may include a visual indication (e.g., a visual object) corresponding to the parameter prioritization list. The visual object may provide an indication of an identified action for the medical provider. For example, the visual object may provide an indication that at least one projected medical parameter is associated with a greatest or least evaluation factor of a set of evaluation factors associated with the plurality of projected medical parameters. The at least one projected medical parameter may be one of the projected medical parameters of the set of projected medical parameters associated with the medical provider. In some embodiments, the visual object may provide an indication associated with each evaluation factor of the one or more evaluation factors. In this way, a graphical display including a visual object may generate an alert (e.g., via processor 104 and/or data visualization tool 112) based on at least one parameter value of at least one projected medical parameter of a medical provider being associated with the greatest or least evaluation factor (e.g., the parameter value is a furthest value from an average parameter values based on a difference between the parameter value and the average parameter value).

In some embodiments, the visual object may include a graph (e.g., plotted points connected with a line, a bar graph, and/or the like) depicting values of the evaluation factors or the parameter values associated with the one or more projected medical parameters representing points on the graph. In some embodiments, the visual object may include an ordered list of the set of projected medical parameters for the medical provider, the ordered list including a name of the projected medical parameter associated (e.g., visually associated) with the parameter value off the projected medical parameter. In some embodiments, the visual object may include color to further distinguish prioritization of projected medical parameters in the visual object and/or to further identify an action of the medical provider.

The indication that the at least one projected medical parameter is associated with a greatest or least evaluation factor of a set of evaluation factors may include a visual indication, such as a color. In some embodiments, the indication may include an ordered list of the set of projected medical parameters for the medical provider, the ordered list including a name of the projected medical parameter with the projected medical parameter marked as first on the ordered list (e.g., shown at the top of the ordered list, and/or the like). In some embodiments, processor 104 may generate a report based on the projected medical parameter associated with the greatest evaluation factor. The report may include details of the projected medical parameter, such that the medical provider associated with the projected medical parameter may view and/or receive the report and data associated with the report.

In some embodiments, processor 104 may transmit a signal to the contact computing system associated with the medical provider and the projected medical parameter associated with the greatest evaluation factor such that the signal causes an alert to be received at the contact computing system. In some embodiments, processor 104 may identify a contact point associated with the at least one projected medical parameter and/or processor 104 may identify the contact computing system associated with the medical provider based on the projected medical parameter (e.g., a type of the projected medical parameter).

In some embodiments, a greatest or least evaluation factor may include a measure of standard deviation from an average parameter value associated with the plurality of projected medical parameters, or a greatest or least evaluation factor may include a standardized score (e.g., a z-score). Various types of evaluation factors may be used to rank and/or prioritize projected medical parameters.

Processor 104 may determine an average parameter value based on averaging each parameter value of a projected medical parameter across a plurality of medical providers. For example, processor 104 may determine an average parameter value associated with a projected medical parameter having a type of catheter-associated urinary tract infection across the medical providers provided in Table 1 (e.g., medical providers 1 through 4). Thus, processor 104 may determine the average parameter value for catheter-associated urinary tract infection by summing 0.92 (medical provider 1), 0.81 (medical provider 2), 0.90 (medical provider 3), and 0.94 (medical provider 4) to determine an intermediate value of 3.57, and by subsequently dividing the intermediate parameter by a number of parameter values (e.g., a number of medical providers) considered by processor 104 to determine the average parameter value being 0.8925. In some embodiments, the number of parameter values considered by processor 104 when determining the average parameter value will typically include all parameter values for a type of projected medical parameter across all medical providers where data associated with medical providers is available.

Processor 104 may determine a standard deviation of each parameter value of the projected medical parameter (e.g., catheter-associated urinary tract infection) based on the average parameter value associated with the projected medical parameters. Processor 104 may determine an average parameter value for each projected medical parameter across any number of medical providers. Processor 104 may determine a standardized score (e.g., a z-score) for each projected medical parameter. A standardized score may include a score based on a number of standard deviations from a mean and/or the average parameter. A standardized score (e.g., a z-score) may represent a number of standard deviations the parameter value of a projected medical parameter is from an average parameter value for the projected medical parameter. For example, a z-score of 2 may represent that the parameter value of the projected medical parameter is 2 standard deviations away from the average value of the projected medical parameter across all medical providers. In some embodiments, processor 104 may determine the standardized score for a projected medical parameter based on the following:

$$standardized score = \frac{( parameter value - average parameter value )}{standard deviation} .$$

In some embodiments, processor 104 may multiply the parameter value of a projected medical parameter by negative one (−1) in instances where a lower parameter value of the projected medical provider is considered to be an indication of higher (e.g., better, more favorable, and/or the like) performance, rather than a higher parameter value. Processor 104 may multiply the parameter value of a projected medical parameter by negative one (−1) prior to determining the standardized score for the projected medical parameter. In this way, the system may set all parameter values of projected medical parameters to a common scale, such that ranking and/or prioritizing projected medical parameters can be done by only considering a magnitude of the evaluation factors.

As an example, consider two projected medical parameters including a first projected medical parameter of Cleanliness and Quietness of Hospital Environment where a higher parameter value indicates better and/or more favorable performance and a second projected medical parameter of Heart Failure 30-Day Mortality Rate where a lower parameter value indicates better and/or more favorable performance. The second projected medical parameter may be multiplied by negative one (−1) such that the second projected medical parameter may be on a common scale with the first projected medical parameter. Thus, both the first and second projected medical parameter will have parameter values where a higher parameter value indicates better and/or favorable performance. In this way, the system may more easily compare evaluation factors on a common scale such that the projected medical parameters may be ranked and/or prioritized without having to consider whether higher or lower values indicate better and/or more favorable performance for the projected medical parameters.

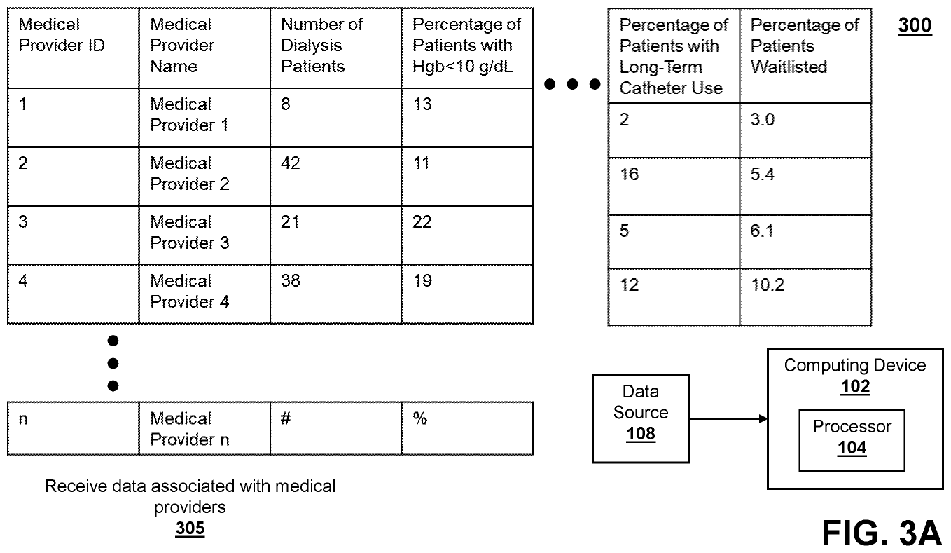

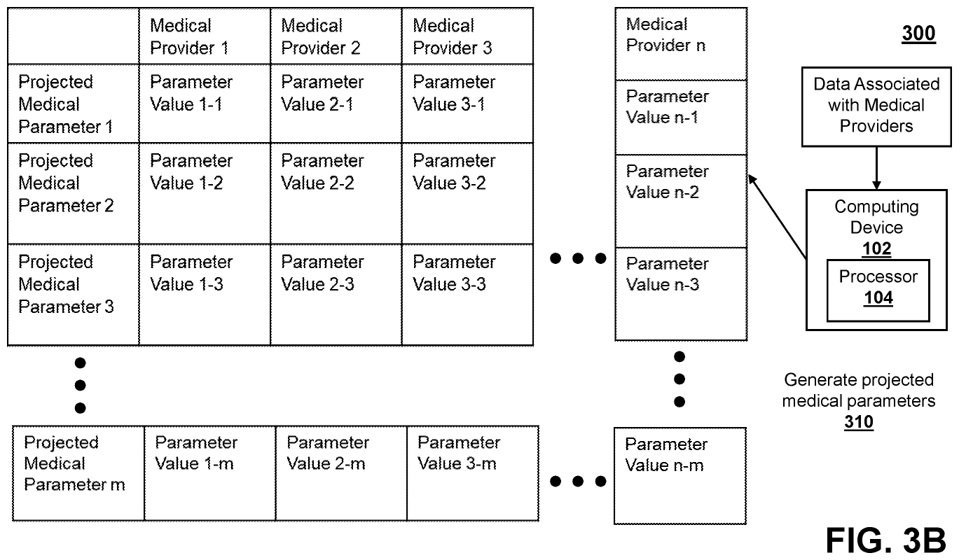

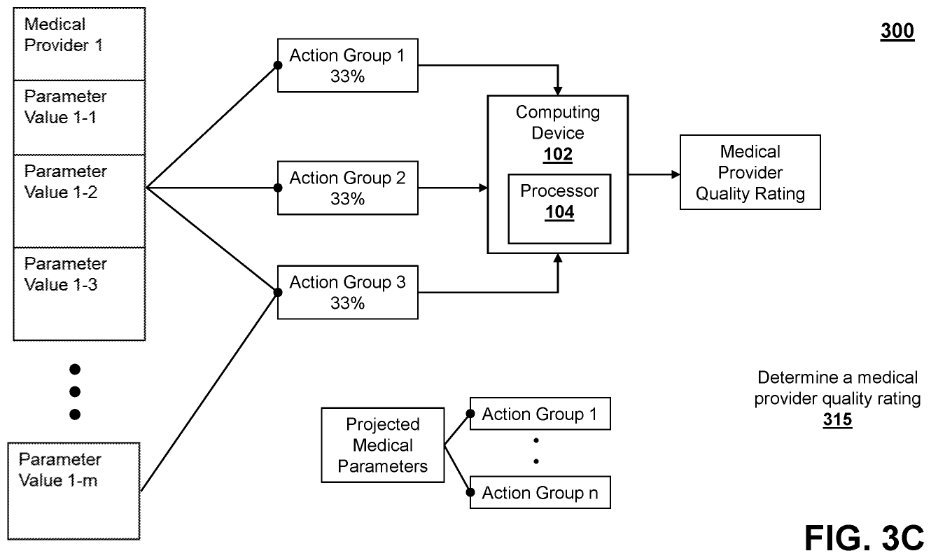

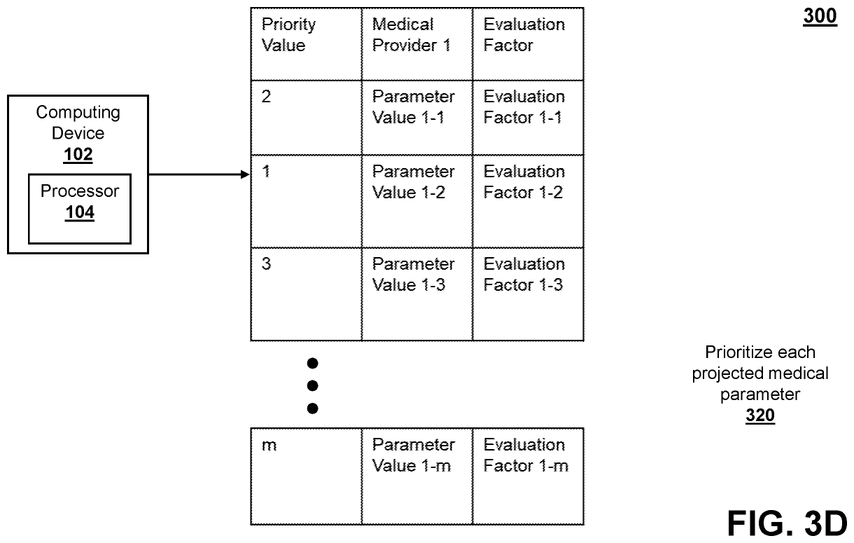

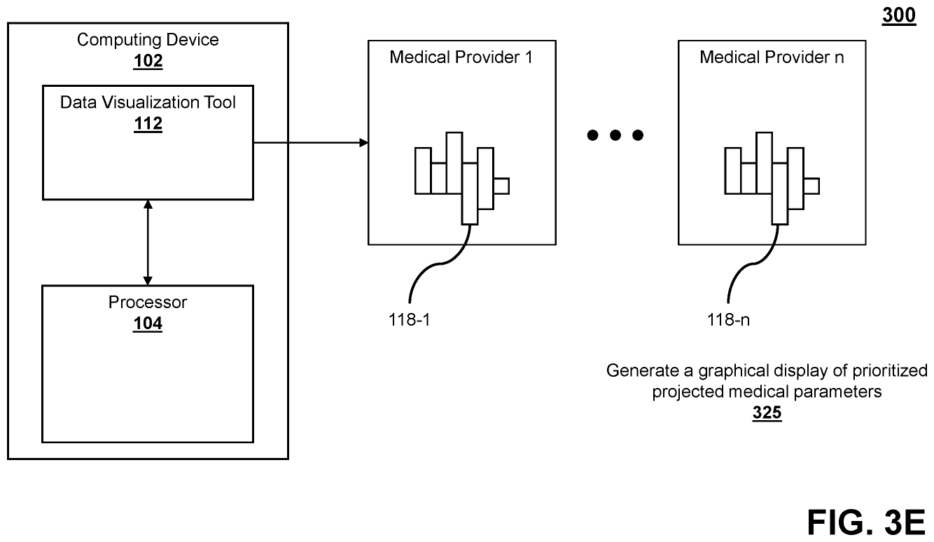

Referring to FIGS. 3A-3E, FIGS. 3A-3E are diagrams of exemplary embodiments of an implementation 300 of a process (e.g., method 200) for identifying actions for medical providers. As shown in FIGS. 3A-3E, implementation 300 may include computing device 102 (and processor 104 thereof) performing the steps of the process.

As shown by reference number 305 in FIG. 3A, computing device 102 (e.g., processor 104 thereof) may receive data associated with medical providers. For example, computing device 102 may receive data associated with medical providers from data source 108. In some embodiments, the data associated with medical providers may include data such as a plurality of medical provider identifiers (IDs), a plurality of medical provider names, a plurality of medical parameter names, and a plurality of values associated with each medical parameter name. The data associated with medical providers may include more data, less data, and/or different data than the data shown in FIG. 3A. The data associated with medical providers shown in FIG. 3A is merely provided as an example.

As shown by reference number 310 in FIG. 3B, computing device 102 (e.g., processor 104 thereof) may generate projected medical parameters. For example, computing device 102 may generate a plurality of projected medical parameters based on the data associated with medical providers received from data source 108. Each projected medical parameter may include a projected medical parameter type (e.g., a name of a projected medical parameter) and a parameter values (e.g., a value assigned to the projected medical parameter type for each medical provider).

As shown in FIG. 3B, each medical provider of a plurality of medical providers may be associated with each projected medical parameter. Each medical provider may have a parameter value associated with each projected medical parameter, where each parameter value is generated by computing device 102 based on data associated with each medical provider and data associated with each projected medical parameter type, where the data associated with each medical provider and data associated with each projected medical parameter type is cross referenced to generate a parameter value for each projected medical parameter and for every medical provider.

As shown by reference number 315 in FIG. 3C, computing device 102 (e.g., processor 104 thereof) may determine a medical provider quality rating. For example, computing device 102 may determine a medical provider quality rating associated with the medical provider based on the projected medical parameters and a plurality of action groups. Each projected medical parameter may be assigned to an action group of the plurality of action groups. Each action group may be assigned a weight value based on a number of action groups that apply to the medical provider. The total weight of the action groups may add to 100%. For example, if the number of action groups that apply to the medical provider is 3, then each action group may have a weight value of 33.3%. Alternatively, a first action group may have a weight of 25%, while a second action group and a third action group each have a weight value of 37.5%. Each projected medical parameter may be assigned the weight value of the action group for which the projected medical parameter has been assigned. The weight values may be taken into account by computing device 102 when determining the medical provider quality rating along with the action group the projected medical provider is assigned to. The weight values may be determined automatically and/or predetermined and may be based on a total number of action groups that apply to the medical provider.

For example, as shown in FIG. 3C, a number of action groups that may apply to medical provider 1 is three action groups (3). Medical provider 1 may be associated with m projected medical parameters. Assuming m is one hundred (100) for medical provider 1, projected medical parameters 1-20 may be assigned to action group 1, projected medical parameters 21-70 may be assigned to action group 2, while projected medical parameters 71-100 may be assigned to action group 3. Thus, each projected medical parameter of projected medical parameters 1-20 may be assigned a weight value of 22% because this is the weight value of action group 1. Additionally, each projected medical parameter of projected medical parameters 21-70 may be assigned a weight value of 39% because this is the weight value of action group 2. Each projected medical parameter of projected medical parameters 71-100 may be assigned a weight value of 39% because this is the weight value of action group 3.

Further, there may exist additional action groups (e.g., action groups 4, 5, and 6) for which medical provider 1 did not report any data. Thus, for medical provider 1, the additional action groups are considered to be “missing”, because none of the projected medical parameters associated with medical provider 1 were assigned to the additional action groups (e.g., medical provider 1 did not report projected medical providers associated with the additional action groups). Thus, when an action group is missing or does not have any projected medical parameters reported for a particular medical provider, the weight values of the additional action groups may be proportioned accordingly across the action groups for which the medical provider does report projected medical parameters (e.g., in FIG. 3C, action group 1, action group 2, and action group 3 ).

For example, the plurality of action groups may include five (5) action groups, where action group 1 through action group 4 may each have a weight value of 22%. Action group 5 may have a weight value of 12%. With reference to FIG. 3C, medical provider 1 reported data for projected medical parameters which are only associated with action group 1 through action group 3. In this example, the weight values of the action groups may be proportioned accordingly across the three action groups. For example, determining a proportioning of weight values across action groups of the plurality of action groups having reported data may involve the following equation:

$$proportioned {weight}_{x} = \frac{{weight}_{x}}{( 100 - \sum {weight}_{y} )} , where y = weight value of missing action group y$$

where proportioned weightₓ is the calculated proportional weight value of each action group, action group x (e.g., action group 1, 2, or 3), weightₓ is the original weight value (e.g., unproportioned weight value) of each action group, action group x, and weight_{y} is each weight value for each missing action group, or each action group that the medical provider did not report data for. Thus, in the example shown in FIG. 3C, the proportioned weight for each action group 1, 2, and 3 would equally be 33% each, as shown in FIG. 3C.

The aforementioned example and the example provided in FIG. 3C is provided only to describe exemplary action groups and an exemplary determination of a medical provider quality rating and should not be considered as limiting the disclosure described herein. It should be understood that various numbers of action groups as well as various weight values may be contemplated.

Computing device 102 may determine the medical provider quality rating for medical provider 1 based on the projected medical parameters associated with medical provider 1 (e.g., the set of projected medical parameters) and based on the weight value assigned to each projected medical parameter of the set of projected medical parameters. As explained previously, weight values may be assigned to projected medical parameters (and determined) based on a number of action groups that the projected medical parameters may be assigned to. The number of action groups required for a set of projected medical parameters may depend on the types of projected medical parameters determined for a medical provider in FIG. 3B, reference number 310.

As shown by reference number 320 in FIG. 3D, computing device 102 (e.g., processor 104 thereof) may prioritize each projected medical parameter. For example, computing device 102 may prioritize each projected medical parameter based on an evaluation factor associated with each projected medical parameter and based on a parameter value assigned to each projected medical parameter to provide a parameter prioritization list. The parameter prioritization list may represent a generated recommendation of one or more projected medical parameters associated with the medical provider that a user (e.g., a user of computing device 102 and/or client device 114) may improve in order to increase a medical provider quality rating.

The evaluation factor may include a difference between the parameter value of the projected medical parameter and an average value associated with the projected medical parameter. In some embodiments, the evaluation factor may include a measure of standard deviation (e.g., a standard deviation value) of the projected medical parameter across a plurality of medical providers.

As shown by reference number 325 in FIG. 3E, computing device 102 (e.g., processor 104 executing data visualization tool 112 thereof) may generate a graphical display of prioritized projected medical parameters. For example, computing device 102 may generate a graphical display associated with the medical provider (e.g., medical provider 1) for transmitting to client display device 116. The graphical display may include one or more visual objects 118-1 through 118-n corresponding to one or more parameter prioritization lists associated with one or more medical providers (e.g., visual object 118-1 may correspond to a first parameter prioritization list associated with medical provider 1). The one or more visual objects 118 may provide an indication of an identified action for the medical provider. For example, the one or more visual objects 118 may provide an indication that at least one projected medical parameter is associated with a greatest or least evaluation factor of a set of evaluation factors.

For example, as shown in FIG. 3E and with reference to FIG. 3D, visual object 118-1 for medical provider 1 may provide an indication that projected medical parameter 1-2 in FIG. 3D is associated with a greatest or least evaluation factor, the greatest or least evaluation factor being evaluation factor 1-2 associated with projected medical parameter 1-2 for medical provider 1. Each medical provider of a plurality of medical providers may be associated with similar visual objects 118 depending on a parameter prioritization list associated with each medical provider. The one or more visual objects 118 may include graphs, images, lists of text, and/or the like displayed on a display device (e.g., client display device 116 of client device 114).

In some embodiments, a client device 114 may include a contact computing system, where the contact computing system is associated with a medical provider. The contact computing system may have exclusive access to a graphical display of visual object 118 associated with the medical provider that has control over the contact computing system. For example, medical provider 1 may include a contact computing system located at a facility of medical provider 1. The contact computing system for medical provider 1 may act as a client device 114 and may be the only contact computing system that may access a graphical display displaying visual object 118-1. In this way, only visual objects 118 that are associated with a specific medical provider can be accessed and viewed by that medical provider.

In some embodiments, the one or more visual objects 118 may be accessible by a plurality of client devices 114 (e.g., a plurality of contact computing systems) such that all client devices 114 receiving the graphical display may access and view visual objects 118. In this way, a plurality of medical providers can access and view data and/or visual objects 118 for other (e.g., peer) medical providers. However, in some embodiments, not all medical providers may be able to access parameter prioritization lists associated with other medical providers.

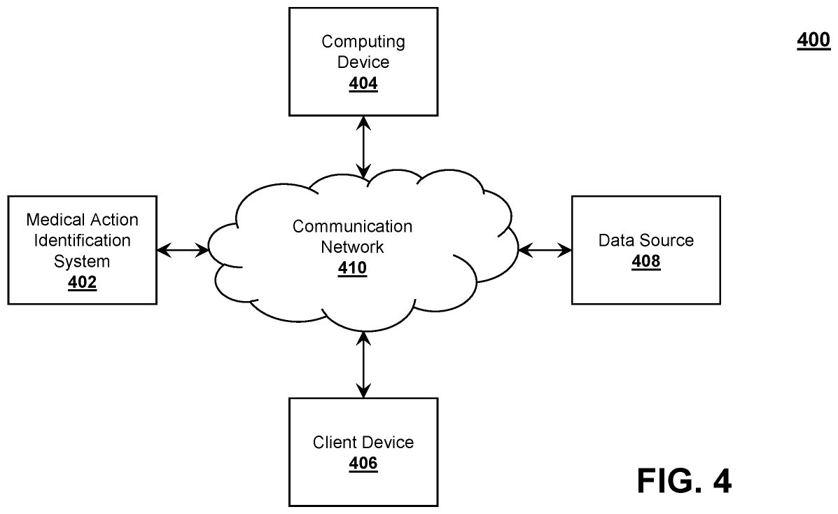

Referring to FIG. 4, shown is a diagram of a non-limiting embodiment of an exemplary environment 400 in which systems, products, and/or methods, as described herein, may be implemented. As shown in FIG. 4, environment 400 may include medical action identification system 402, computing device 404, client device 406, data source 408, and communication network 410. In some embodiments, each of computing device 404, client device 406, data source 408, and/or communication network 410 may be implemented by (e.g., part of) medical action identification system 402. In some embodiments, at least one of each of computing device 404, client device 406, data source 408, and/or communication network 410 may be implemented by (e.g., part of) another system, another device, another group of systems, or another group of devices, separate from or including medical action identification system 402, such as computing device 404, client device 406, data source 408, and/or the like.

Medical action identification system 402 may include one or more devices capable of receiving information from and/or communicating information to computing device 404, client devices 406, and/or data source 408 via communication network 410. For example, medical action identification system 402 may include a computing device (e.g., computing device 102), such as a server, a group of servers, and/or other like devices. In some non-limiting embodiments or aspects, medical action identification system 402 may be associated with a server as described herein. In some non-limiting embodiments or aspects, medical action identification system 402 may be in communication with a data storage device (e.g., database, memory, shared memory, cache memory, data source 408, and/or the like), which may be local or remote to medical action identification system 402. In some embodiments, medical action identification system 402 may be capable of receiving information from, storing information in, communicating information to, and/or searching information stored in the data storage device (e.g., data source 408). Medical action identification system may be the same as or similar to computing device 102.

Computing device 404 may include one or more devices capable of receiving information and/or communicating information to medical action identification system 402, client device 406, and/or data source 408 via communication network 410. For example, computing device 404 may include a computing device, such as a server, a group of servers, and/or other like devices. In some embodiments, computing device 404 may be associated with a server, a client device, and/or a user device as described herein. In some embodiments, computing device 404 may be the same as or similar to computing device 102. In some embodiments, computing device 404 may include a contact computing system as described herein.

Client device 406 may include one or more devices capable of receiving information from and/or communicating information to medical action identification system 402, computing device 404, and/or data source 408 via communication network 410. Additionally or alternatively, one or more client devices 406 may include a device capable of receiving information from and/or communicating information to other client devices 406 via communication network 410, another network (e.g., an ad hoc network, a local network, a private network, a virtual private network, and/or the like), and/or any other suitable communication technique. For example, client device 406 may include a user device and/or the like. Client device 406 may be the same as or similar to client device 114.

Communication network 410 may include one or more wired and/or wireless networks. For example, communication network 410 may include a cellular network (e.g., a long-term evolution (LTE®) network, a third generation (3G) network, a fourth generation (4G) network, a fifth generation (5G) network, a code division multiple access (CDMA) network, and/or the like), a public land mobile network (PLMN), a local area network (LAN), a wide area network (WAN), a metropolitan area network (MAN), a telephone network (e.g., the public switched telephone network (PSTN)), a private network (e.g., a private network associated with medical action identification system 402), an ad hoc network, an intranet, the Internet, a fiber optic-based network, a cloud computing network, and/or the like, and/or a combination of these or other types of networks.

The number and arrangement of systems, devices, and/or networks shown in FIG. 4 are provided as an example. There may be additional systems, devices, and/or networks; fewer systems, devices, and/or networks; different systems, devices, and/or networks; and/or differently arranged systems, devices, and/or networks than those shown in FIG. 4. Furthermore, two or more systems or devices shown in FIG. 4 may be implemented within a single system or device, or a single system or device shown in FIG. 4 may be implemented as multiple, distributed systems or devices. Additionally or alternatively, a set of systems (e.g., one or more systems) or a set of devices (e.g., one or more devices) of environment 400 may perform one or more functions described as being performed by another set of systems or another set of devices of environment 400.

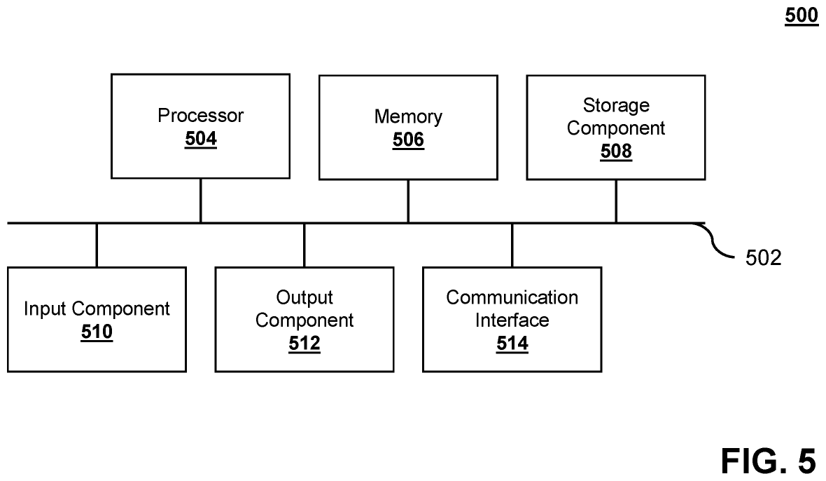

Referring to FIG. 5, shown is a diagram of example components of a device 500 according to non-limiting embodiments or aspects. Device 500 (and/or at least one component of device 500) may correspond to at least one of computing device 102, processor 104, memory 106, data source 108, database 110, client device 114, and/or client display device 116 in FIG. 1 and/or at least one of medical action identification system 402, computing device 404, client device 406, and/or data source 408 in FIG. 4, as an example. In some embodiments, such systems or devices in FIG. 1 or FIG. 4 may include at least one device 500 and/or at least one component of device 500. The number and arrangement of components shown in FIG. 5 are provided as an example. In some embodiments, device 500 may include additional components, fewer components, different components, or differently arranged components than those shown in FIG. 5. Additionally, or alternatively, a set of components (e.g., one or more components) of device 500 may perform one or more functions described as being performed by another set of components of device 500.

As shown in FIG. 5, device 500 may include bus 502, processor 504, memory 506, storage component 508, input component 510, output component 512, and communication interface 514. Bus 502 may include a component that permits communication among the components of device 500. In some embodiments, bus 502 may not implement a hardware cache coherence protocol. In some embodiments, processor 504 may be implemented in hardware, software (e.g., firmware), or a combination of hardware and software. For example, processor 504 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. In some embodiments, processor 504 may be the same as or similar to processor 104.

Memory 506 may include random access memory RAM, ROM, and/or another type of dynamic or static storage device (e.g., flash memory, magnetic memory, optical memory, etc.) that stores information and/or instructions for use by processor 504. In some embodiments, memory 506 may be the same as or similar to memory 106.

With continued reference to FIG. 5, storage component 508 may store information and/or software related to the operation and use of device 500. For example, storage component 508 may include a hard disk (e.g., a magnetic disk, an optical disk, a magneto-optic disk, a solid state disk, etc.) and/or another type of computer-readable medium. Storage component 508 may store information and/or software related to the operation and use of device 500 such as data visualization tool 112. In some embodiments, storage component 508 may be the same as or similar to memory 106.

Input component 510 may include a component that permits device 500 to receive information, such as via user input (e.g., a touch screen display, a keyboard, a keypad, a mouse, a button, a switch, a microphone, etc.). Additionally, or alternatively, input component 510 may include a sensor for sensing information (e.g., a global positioning system (GPS) component, an accelerometer, a gyroscope, an actuator, etc.).

Output component 512 may include a component that provides output information from device 500 (e.g., a display such as client display device 116, a speaker, one or more light-emitting diodes (LEDs), etc.). Output component 512 may include a component of client device 114 (e.g., a contact computing system) that may provide an alert to another system and/or a user.

Communication interface 514 may include a transceiver-like component (e.g., a transceiver, a receiver, a transmitter, a separate receiver and transmitter pair, etc.) that enables device 500 to communicate with other devices, such as via a wired connection, a wireless connection, or a combination of wired and wireless connections. Communication interface 514 may permit device 500 to receive information from another device and/or provide information to another device. For example, communication interface 514 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 500 may perform one or more methods and/or processes described herein. Device 500 may perform these processes based on processor 504 executing software instructions stored by a computer-readable medium, such as memory 506 and/or storage component 508. A computer-readable medium may include any non-transitory memory device. A memory device includes memory space located inside of a single physical storage device or memory space spread across multiple physical storage devices.

Software instructions (e.g., software instructions corresponding to data visualization tool 112) may be read into memory 506 and/or storage component 508 from another computer-readable medium, or from another device via communication interface 514. When executed, software instructions stored in memory 506 and/or storage component 508 may cause processor 504 to perform one or more processes described herein. Additionally or alternatively, hardwired circuitry may be used in combination with software instructions to perform one or more processes described herein. Thus, embodiments described herein are not limited to any specific combination of hardware circuitry and software. The term “programmed or configured,” as used herein, refers to an arrangement of software, hardware circuitry, or any combination thereof on one or more devices.

Any of the processors (e.g., processor 104 and/or processor 504) disclosed herein may be part of or in communication with a machine (e.g., a computing device such as computing device 102, a logic device, a circuit, an operating module (hardware, software, and/or firmware), etc.). The processor may be hardware (e.g., processor, integrated circuit, central processing unit, microprocessor, core processor, computer device, etc.), firmware, software, etc. configured to perform operations by execution of instructions embodied in computer program code, algorithms, program logic, control, logic, data processing program logic, artificial intelligence programming, machine learning programming, artificial neural network programming, automated reasoning programming, etc. The processor may receive, process, and/or store data related to sensors and/or network communications (e.g., over communication network 410), for example.

Any of the processors (e.g., processor 104 and/or processor 504) disclosed herein may be a scalable processor, a parallelizable processor, a multi-thread processing processor, etc. The processor may be a computer in which the processing power is selected as a function of anticipated network traffic. The processor may include any integrated circuit or other electronic device (or collection of devices) capable of performing an operation on at least one instruction, which can include a Reduced Instruction Set Core (RISC) processor, a CISC microprocessor, a Microcontroller Unit (MCU), a CISC-based Central Processing Unit (CPU), a Digital Signal Processor (DSP), a Graphics Processing Unit (GPU), a Field Programmable Gate Array (FPGA), etc. The hardware of such devices may be integrated onto a single substrate (e.g., silicon “die”), or distributed among two or more substrates. Various functional aspects of the processor may be implemented solely as software or firmware associated with the processor.

The processor may include one or more processing or operating modules. A processing or operating module may include a software or firmware operating module configured to implement any of the functions disclosed herein. The processing or operating module may be embodied as software and stored in memory, the memory being operatively associated with the processor. A processing module may be embodied as a web application, a desktop application, a console application, and/or the like.

The processor may include and/or be associated with a computer (e.g., computing device 102) or machine-readable medium. The computer or machine-readable medium may include memory (e.g., memory 106). Any of the memory discussed herein may be computer readable memory configured to store data. The memory may include a volatile or non-volatile, transitory or non-transitory memory, and may be embodied as an in-memory, an active memory, a cloud memory, etc. Examples of memory can include flash memory, Random Access Memory (RAM), Read Only Memory (ROM), Programmable Read only Memory (PROM), Erasable Programmable Read only Memory (EPROM), Electronically Erasable Programmable Read only Memory (EEPROM), FLASH-EPROM, Compact Disc (CD)-ROM, Digital Optical Disc DVD), optical storage, optical medium, a carrier wave, magnetic cassettes, magnetic tape, magnetic disk storage or other magnetic storage devices, or any other medium which may be used to store the desired information and which may be accessed by the processor.

Any of the processors (e.g., processor 104 and/or processor 504) may be in communication with other processors of other devices (e.g., a computing device such as computing device 102 and/or client device 114, a computer system, a laptop computer, a desktop computer, etc.). For instance, processor 104 of computing device 102 may be in communication with the processor of client device 114, etc. Any of the processors may have transceivers or other communication devices/circuitry to facilitate transmission and reception of wireless signals. Any of the processors may include an application programming interface (API) as a software intermediary that allows two or more applications to communicate and/or transmit information between each other.

The memory may be a non-transitory computer-readable medium. The term “computer-readable medium” (or “machine-readable medium”) as used herein is an extensible term that refers to any medium or any memory, that participates in providing instructions to the processor for execution, or any mechanism for storing and/or transmitting information in a form readable by a machine (e.g., a computer, a computing device, etc.). Such a medium may store computer-executable instructions to be executed by a processing element and/or control logic, and data which is manipulated by a processing element and/or control logic, and may take many forms, including but not limited to, non-volatile medium, volatile medium, transmission media, etc. The computer or machine-readable medium may be configured to store one or more instructions thereon. The instructions may be in the form of algorithms, program logic, etc. that cause the processor to execute any of the functions disclosed herein.

Embodiments of the memory may include a processor module and other circuitry to allow for the transfer of data to and from the memory, which may include to and from other components of a communication system. This transfer can be via hardwire or wireless transmission. The communication system may include transceivers, which can be used in combination with switches, receivers, transmitters, routers, gateways, wave-guides, etc. to facilitate communications via a communication approach or protocol for controlled and/or coordinated signal transmission and processing to any other component or combination of components of the communication system. The transmission can be via a communication link. The communication link may be electronic-based, optical-based, opto-electronic-based, quantum-based, etc. Communications may be via Bluetooth, near field communications, cellular communications, telemetry communications, Internet communications, etc.

Data stored in the exemplary computing device (e.g., in the memory, such as memory 106 of computing device 102) may be stored on any type of suitable computer-readable media, such as optical storage (e.g., a compact disc, digital versatile disc, Blu-ray disc, etc.), magnetic tape storage (e.g., a hard disk drive), or solid-state drive. An operating system may also be stored in the memory.

In an exemplary embodiment, the data can be configured in any type of suitable database configuration (e.g., in database 110), such as a relational database, a structured query language (SQL) database, a distributed database, an object database, etc. Suitable configurations and storage types will be apparent to persons having skill in the relevant art.

The exemplary computing device (e.g., computing device 102) can also include a communications interface (e.g., communication interface 514). The communications interface may be configured to allow software and data to be transferred between the computing device and external devices. Exemplary communications interfaces can include a modem, a network interface (e.g., an Ethernet card), a communications port, a PCMCIA slot and card, etc. Software and data transferred via the communications interface may be in the form of signals, which may be electronic, electromagnetic, optical, or other signals as will be apparent to persons having skill in the relevant art. The signals may travel via a communications path, which can be configured to carry the signals and may be implemented using wire, cable, fiber optics, a phone line, a cellular phone link, a radio frequency link, etc. Transmission of data and signals can be via transmission media. Transmission media can include coaxial cables, copper wire, fiber optics, etc. Transmission media may also take the form of acoustic or light waves, such as those generated during radio-wave and infrared data communications, or other form of propagated signals (e.g., carrier waves, digital signals, etc.).

Memory semiconductors (e.g., DRAMs, etc.) may be means for providing software to the computing device. Computer programs (e.g., computer control logic) may be stored in the memory. Computer programs may also be received via the communications interface. Such computer programs, when executed, may enable a computing device to implement the present methods as discussed herein. In particular, the computer programs stored on a non-transitory computer-readable medium, when executed, may enable a hardware processor device to implement the methods as discussed herein. Accordingly, such computer programs can represent controllers of the computing device.

Where the present disclosure is implemented using software, the software may be stored in a computer program product or non-transitory computer readable medium and loaded into the computing device using a removable storage drive or communications interface. In an exemplary embodiment, any computing device disclosed herein may also include a display interface that outputs display signals to a display unit and/or display device, e.g., LCD screen, plasma screen, LED screen, DLP screen, CRT screen, etc.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

What is claimed is:

1. A system for identifying actions for medical providers, comprising:
a computing device including memory storing program instructions for a data visualization tool and program instructions for a medical provider quality algorithm, the computing device including at least one processor programmed or configured to:
execute the data visualization tool;
receive data associated with medical providers; and
execute the medical provider quality algorithm to generate a plurality of projected medical parameters and a medical provider quality rating;
a database configured to store the data associated with medical providers, the plurality of projected medical parameters, and the medical provider quality rating; and
the data visualization tool configured to generate a graphical display including a visual object corresponding to a parameter prioritization list based on a set of evaluation factors associated with a set of projected medical parameters of the plurality of projected medical parameters, the visual object providing an indication of an identified action for a medical provider.

2. The system of claim 1, wherein the indication of an identified action for the medical provider comprises an indication that at least one projected medical parameter of the set of projected medical parameters is associated with a least evaluation factor of the set of evaluation factors, wherein the data visualization tool is configured to transmit the graphical display to at least one client device including a client display device.

3. The system of claim 1, wherein the data associated with medical providers is received from a medical provider data source.

4. The system of claim 1, wherein each evaluation factor of the set of evaluation factors is a standardized score.

5. The system of claim 1, wherein the at least one processor is further programmed or configured to:
aggregate each projected medical parameter and each evaluation factor, wherein the set of projected medical providers and the set of evaluation factors is associated with the medical provider; and
rank each projected medical parameter for the medical provider based on the parameter value associated with the projected medical parameter to provide ranked medical parameters.

6. The system of claim 1, wherein the at least one processor is further programmed or configured to:
determine an average parameter value based on averaging each parameter value of a type of a projected medical parameter across a plurality of medical providers.

7. The system of claim 1, wherein the at least one processor is further programmed or configured to:
identify a contact point associated with at least one projected medical parameter.

8. The system of claim 5, wherein the at least one processor is further programmed or configured to:
assign a ranked medical parameter a higher priority value where the evaluation factor associated with the ranked medical parameter is less than all remaining evaluation factors of the set of evaluation factors; and
assign the ranked medical parameter a lower priority value where the evaluation factor associated with the ranked medical parameter is greater than all the remaining evaluation factors of the set of evaluation factors.

9. The system of claim 1, wherein each evaluation factor is a value equal to a difference between the projected medical parameter and an average parameter value associated with the projected medical parameter.

10. A computer-implemented method for identifying actions for medical providers, comprising:
receiving, with at least one processor, data associated with medical providers;
generating, with the at least one processor, a plurality of projected medical parameters including a parameter value based on the data associated with medical providers, wherein a set of projected medical parameters of the plurality of projected medical parameters is associated with a medical provider;
determining, with the at least one processor, a medical provider quality rating associated with the medical provider based on the set of projected medical parameters and a plurality of action groups, each projected medical parameter of the set of projected medical parameters being assigned to an action group of the plurality of action groups, each projected medical parameter having a weight value based on a number of action groups that apply to the medical provider;
prioritizing, with the at least one processor, each projected medical parameter based on an evaluation factor of a set of evaluation factors associated with the set of projected medical parameters and based on the parameter value to provide a parameter prioritization list, the parameter prioritization list representing a generated recommendation of the set of projected medical parameters for a user to improve to increase the medical provider quality rating; and
generating, with the at least one processor, a graphical display associated with the medical provider for transmitting to a client display device, the graphical display including a visual object corresponding to the parameter prioritization list, the visual object providing an indication of an identified action for the medical provider.

11. The computer-implemented method of claim 10, wherein the indication of an identified action for the medical provider comprises an indication that at least one projected medical parameter of the set of projected medical parameters is associated with a least evaluation factor of the set of evaluation factors.

12. The computer-implemented method of claim 10, further comprising:
transmitting the graphical display to at least one client device including a client display device.

13. The computer-implemented method of claim 10, wherein the data associated with medical providers is received from a medical provider data source.

14. The computer-implemented method of claim 10, wherein a plurality of medical providers includes the medical provider, each other medical provider of the plurality of medical providers being associated with a different set of projected medical parameters of the plurality of projected medical parameters.

15. The computer-implemented method of claim 10, wherein each evaluation factor of the set of evaluation factors is a standardized score.

16. The computer-implemented method of claim 10, wherein prioritizing each projected medical parameter comprises:
aggregating each projected medical parameter and each evaluation factor; and
ranking each projected medical parameter for the medical provider based on the evaluation factor associated with the projected medical parameter to provide ranked medical parameters.

17. The computer-implemented method of claim 11, wherein the least evaluation factor includes a value representing a number of standard deviations from an average parameter value of a projected medical parameter, the average parameter value being determined based on averaging each parameter value of a type of the projected medical parameter across a plurality of medical providers.

18. The computer-implemented method of claim 10, further comprising:
identifying a contact point associated with at least one projected medical parameter.

19. The computer-implemented method of claim 16, wherein prioritizing each ranked medical parameter further comprises:
assigning a ranked medical parameter a higher priority value where the evaluation factor associated with the ranked medical parameter is less than all remaining evaluation factors of the set of evaluation factors; and
assigning the ranked medical parameter a lower priority value where the evaluation factor associated with the ranked medical parameter is greater than all the remaining evaluation factors of the set of evaluation factors.

20. The computer-implemented method of claim 10, wherein each evaluation factor is a value equal to a difference between the projected medical parameter and an average parameter value associated with the projected medical parameter.
